(19) United States
(12) Patent Application Publication
(10) Pub. No.: US 2026/0355488 A1
(43) Pub. Date: Nov. 12, 2026

(54) NEUROSURGICAL GUIDE TUBE

(71) Applicant: NEUROCHASE TECHNOLOGIES LIMITED, Bristol (GB)

(72) Inventors: Steven Streatfield GILL, Clifton, Bristol (GB); Thomas GILL, Clifton, Bristol (GB)

(73) Assignee: NEUROCHASE TECHNOLOGIES LIMITED, Bristol (GB)

(21) Appl. No.: 19/123,960

(22) PCT Filed: Oct. 18, 2023
(86) PCT No.: PCT/GB2023/052701

§ 371 Date: Apr. 24, 2025

(30) Foreign Application Priority Data
Oct. 25, 2022 (GB) 2215760.6

(51) Int. Cl. A61M 39/02; A61M 25/00; A61M 25/01; A61M 25/06

(52) U.S. Cl. CPC A61M 39/0247; A61M 25/007; A61M 25/0097; A61M 25/0113; A61M 2025/0042; A61M 2025/0046; A61M 2025/006; A61M 2025/0687; A61M 2039/0205; A61M 2039/025; A61M 2039/0261; A61M 2039/0276; A61M 2205/3344; A61M 2210/0693

(57) ABSTRACT

A guide tube (4) for use with a fluid transfer tube for providing fluid access to the brain of a mammal. The guide tube (4) is configured for insertion into the brain, and comprises: a through-bore for passage of a fluid transfer tube; and a distal end (18) comprising a sealing region (70) configured to compress surrounding brain tissue to form a reflux-inhibiting seal around the distal end (18) of the guide tube, the sealing region comprising a reduced-pressure region (72) in which the compression of the brain tissue is lower than the compression of brain tissue in both a proximally adjacent region and a distally adjacent region. Also disclosed are neurosurgical apparatuses comprising the guide tube and a method for convection-enhanced delivery of an infusate to the brain parenchyma using the guide tube.

### FIELD

The present invention relates to apparatuses and methods for use in neurosurgery. Particularly, the invention relates to an apparatus used to deliver, and a method of delivering, therapeutic agents by infusion directly into the brain parenchyma.

### BACKGROUND

Treatment of neurological diseases can be hindered by the presence of the blood-brain barrier. The blood-brain barrier can make it difficult to develop therapeutic agents that can be delivered from the systemic circulation into the brain parenchyma. It can be desirable to deliver therapeutic agents to specific regions of the brain (‘brain volumes’ or ‘target volumes’). Obtaining an appropriate concentration of the therapeutic agent in a target volume whilst minimising exposure of the rest of the brain to the therapeutic agent is desirable, to reduce undesired side effects.

Convection Enhanced Delivery (CED) is a method of targeted delivery of therapeutic agents to particular brain volumes by the controlled infusion of the therapeutic agent delivered into the brain parenchyma in a fluid using extremely small cannulas or tubing (often referred to in the art as micro-catheters). The cannulas have a port or ports at their distal end, allowing an infusate including the therapeutic agent to exit the catheter into the target brain volume. A continuous pressure gradient must be achieved at the port to overcome the local pressure of the target brain volume, thereby allowing the infusate to flow effectively into the target brain volume.

There are challenges in using CED because fluid flowing from a cannula port will follow the path of least resistance. Typically, the path of least resistance will be back along the cannula/tissue interface, causing so-called reflux, instead of driving the fluid into the tissue as desired. To control reflux, so that fluid is driven into the tissue of the target volume, CED cannulas typically have an abrupt change in diameter (a ‘step’) towards the distal end. For example, the distal end of the cannula may be of less than 1 mm in diameter for a short length to the extreme distal end, with a step up to a diameter of 1.5 to 2.5 mm for the remainder of the cannula. The small-diameter distal end, if inserted with minimal trauma, creates a tissue seal around the cannula, minimising reflux along the interface. The change in diameter at the step compresses tissue on insertion into the brain and further resists reflux.

Careful control of flow rates, typically in the range of 3-5 microlitres per minute, facilitates flow of an infusate into the tissues in preference to reflux. Higher flow rates will tend to increase reflux, and may lead to refluxing infusate getting past the step and flowing back along the cannula/brain tissue interface, even at the wider diameter portion of the cannula. When the step is overwhelmed in this way, the infusate enters a low-resistance pathway through the larger circumferential space around the larger diameter part of the cannula, with a resultant reduction of the intended distribution of infusate within the target volume.

The design of the reflux-resistant step differs between CED cannulas. The cannulas described in EP1482851B1 and US2010/0217228A1 provide a single step. WO2007/024841A2 provides a cannula with a plurality of steps, and WO2014/016591 has a recessed step.

In addition to the step design, the length that a cannula extends beyond the step, to its extreme distal end, is an important determinant of both the volume and shape of distribution of infusate into brain tissue. A short-stepped cannula (i.e., a cannula with only a short length of smaller diameter) will have a small volume of distribution (Vd) that is roughly spherical. As the length of the smaller-diameter portion increases, the Vd becomes larger and more ovoid, then cylindrical, and then pear-shaped with a bulbous distal end. When the radius of distribution of fluid convected into the brain exceeds that of the region of compressed tissue at the step then the infusate will tend to enter the low resistance pathway along the larger diameter portion of the cannula and reflux away from the target volume. This places a limit on the Vd at the target.

To deliver therapy to clinically meaningful tissue volumes with a fixed step-length arrangement can therefore be problematic. Using a cannula with a short, fixed step-length will require sequential infusions made at different points along a trajectory into the brain to fill an elongate volume of tissue. Similarly, multiple passes are required to fill a larger, more spherical structure in the brain. Conversely, filling a smaller target using a catheter with a relatively long ‘step length’ may make it difficult to contain the therapy in the target. Therefore, the ability to adjust the step length for each target provides significant advantages.

Most targets for treating CNS (central nervous system) diseases by CED require several cannulas to be implanted to achieve the desired coverage of infusate into brain tissue. When the desired target volume and shape has been defined from MRI images, the number and orientation of cannulas required to fill the volume are determined by understanding the likely distribution shape and volume that can be achieved with the cannulas being deployed. It is also important to confirm that the infused therapy has covered the prescribed treatment volume with MRI imaging either during or immediately following the infusion.

When rigid cannulas are used, they are fixed in a stereotactic frame whilst the infusion is carried out. In such arrangements the stereotactic frame needs to be MRI-compatible and have a low profile to fit in the imaging coil. Typically, additional cannula placements and infusions are required after completion of the first. Thus, such procedures are relatively lengthy and can expose the patient to increased risks from prolonged anaesthesia and immobilisation.

Alternative procedures employing multiple cannula trajectories make use of flexible cannulas that are implanted and secured to the skull. The flexible cannula tubing extending out of the skull can be attached to a low-profile skull fixation. This facilitates safe transfer into an MRI scanner where simultaneous infusions can be conducted with the patient awake for neurological evaluation. A stereotactic frame is not required during infusion where flexible cannulas are employed. The flexible cannulas may be removed after the infusions are complete, or in some instances may be left in-situ for repeated infusions days, weeks or months later.

Uses of flexible cannulas are described in EP1482851B1, EP2601997A1, EP2819739B1, and WO2014/016591. In these arrangements, the distance between the cannula's distal end and the reflux-resistant step can be adjusted. In each case, the flexible cannula has a proximal hub and is cut to a desired length for insertion to a target point in the brain. When inserted into an implanted guide tube, also cut to a desired length, the cannula's hub acts as a stop when it engages with a proximal head on the guide tube that is fixed in the skull. The cannula extends beyond the guide tube, and the step created by the change in diameter from the cannula to the guide tube provides resistance to reflux of infusate.

In typical procedures to implant flexible cannulas described in EP 1482851B1, EP260 1 997A1 and EP2819739B1, a profiled hole is made in the skull along the selected trajectory, guided by a stereo guide or image guided robot. A probe is then passed through the hole to the planned distal end of the guide tube and withdrawn, leaving a track in the brain tissue. The guide tube, cut to length, is placed over a delivery probe such that the rounded tip of the probe extends just beyond its distal end. The guide tube (on the delivery probe) is inserted down the pre-made track until the head on its proximal end press fits into the formed hole in the skull. The probe is then advanced to the planned position of the cannula target and withdrawn, leaving a track through the tissue that is contiguous with the bore of the guide tube.

The cannula, connected to an infusion pump and delivering infusate at a low flow rate, is inserted down the guide tube so that it emerges from the distal end of the guide tube and passes into and through the pre-formed track in the tissue. The cannula is filled with infusate as it is passed into the brain and the continued slow infusion provided to the cannula prevents coring of tissue during its transit. When a therapeutic fluid is delivered through the cannula's distal port it will generally follow the path of least resistance and flow back along the cannula-tissue interface before meeting the region of tissue compressed by the distal end of the guide tube. The localised pressure at the interface acts to inhibit further reflux and the infusate is then preferentially driven radially into the tissue.

The devices described above have potential disadvantages. The method of insertion can cause micro trauma to the tissues, creating a low resistance path for infusate which impairs reflux control at the guide tube-tissue interface. As the guide tube is inserted into the pre-made track in the brain, the cut edges of its distal end may tend to shear the tissue creating a circumferential column of fragmented tissue. The force required to push fit the head of the guide tube/delivery probe into the preformed hole in the skull is also transferred to the distal end of the guide tube/delivery probe, which may add to the localised tissue trauma in the region of the step.

Additionally, as the cannula is inserted down the guide tube it will tend to act as a piston and drive a column of air ahead of it. Even with the application of suction at the proximal end of the guide tube it is difficult to vent air through the narrow space between the cannula and the guide tube. If air is driven into the brain, it tears the tissue and creates a space-occupying lesion close to the reflux-controlling step. In the short term, this is likely to disrupt the intended pattern of distribution of the infusate, but as the air is absorbed the cavity left can provide a low resistance pathway at the step, which will augment undesired reflux.

EP3119310B1 describes a guide tube with an internal profile configured to provide a fluid return path for carrying any fluid displaced from within the guide tube during insertion of a catheter. Such a path may also conduct and vent air during catheter insertion. However, to create a profile with internal channels of sufficient size to facilitate air venting increases the overall dimensions of the guide tube. A wider diameter guide tube may cause more trauma on insertion. Narrow channels between the guide tube and cannula may also be liable to obstruction of air flow by the presence of liquid, due to surface tension.

International Patent Application WO2014/016591A1 discloses a recessed step arrangement, wherein the guide tube comprises an internal recess that compresses tissue therein. The internal recess provides a step feature that is intended to provide more effective compression of tissue to limit the flow of infusate along the cannula-tissue interface. However, in such an arrangement the guide tube cores a portion of brain tissue on insertion. The local tissue trauma could result in neurological deficits if in an eloquent part of the brain or may cause haemorrhage.

A further potential difficulty with known CED devices, particularly those that are chronically implanted, is brain movement. The brain moves within the skull so that fixing of a cannula and/or associated guide tubes to the skull can cause movement of the tubing or cannula relative to brain tissue. This may cause local tissue trauma, particularly at a reflux resistant step, creating local vacuolation and the creation of a low-resistance pathway that will tend to augment, rather than resist, reflux.

In view of the aforementioned difficulties, there is still a need for a CED apparatus with improved reflux resistance and reduced trauma in use.

### SUMMARY

According to a first aspect, there is provided a guide tube for use with a fluid transfer tube for providing fluid access to the brain of a mammal, wherein the guide tube is configured for insertion into the brain, and the guide tube comprises: a through-bore for passage of a fluid transfer tube; and a distal end comprising a sealing region configured to compress surrounding brain tissue to form a reflux-inhibiting seal around the distal end of the guide tube, the sealing region comprising a reduced-pressure region in which the compression of the brain tissue is lower than the compression of brain tissue in both a proximally adjacent region and a distally adjacent region.

The reduced-pressure region provides an improved sealing effect at the distal end of the guide tube by creating a region in which refluxing fluid is trapped, and requiring fluid to bypass multiple regions of higher compression to reflux up the outer surface of the guide tube. Thereby, there is no need to provide a large step in the outer diameter of the guide tube, and reflux resistance can be provided with a guide tube that has a smaller outside diameter and is thus less traumatic to the brain tissue than currently available reflux-resistant cannulas.

Optionally, the sealing region comprises a plurality of reduced-pressure regions. Multiple reduced-pressure regions further increases the effectiveness of the seal provided at the distal end of the guide tube, and reduces the risk of reflux.

Optionally, the plurality of reduced-pressure regions are equally-spaced along a length of the guide tube. This provides a regular structure and consistent changes in compression along the guide tube.

Optionally, a spacing between the reduced-pressure regions is at most 2 mm, preferably at most 1.5 mm, more preferably at most 1 mm. Having a maximum spacing between regions reduces the length of the guide tube over which fluid is likely to reflux.

Optionally, the reduced-pressure regions are provided over at least 5% of a length of the guide tube. Optionally, the reduced-pressure regions are provided over at least 2 mm of a length of the guide tube.

Providing the regions over a minimum length of the guide tube ensures an adequate minimum sealing performance.

Optionally, at least one of the reduced-pressure regions is at most 20 mm away from an extreme distal end of the guide tube. Optionally, at least one of the reduced-pressure regions is at most 25% of a length of the guide tube away from an extreme distal end of the guide tube. Providing the reduced-pressure region within a certain distance of the distal end ensures that fluid cannot reflux too far up the length of the guide tube.

Optionally, the reduced-pressure region is provided by a groove in an outer surface of the guide tube or between protrusions from an outer surface of the guide tube. A groove or protrusions provide a convenient and readily manufactured way to provide the variations in pressure along the outer surface of the guide tube.

Optionally, the groove has a depth of at most 0.5 mm, or the protrusions have a height of at most 0.5 mm. Optionally, the groove has a depth of at least 0.1 mm, or the protrusions have a height of at least 0.1 mm. This range of dimensions creates a sufficient change in the compression of surrounding tissue to provide good sealing performance to prevent reflux.

Optionally, the groove or protrusions extend around the entire circumference of the guide tube. This provides good sealing protection against reflux at all points around the guide tube, thereby further minimising reflux.

Optionally, an outer surface of the guide tube is configured to make contact with brain tissue following insertion of the guide tube into the brain. This ensures a close seal between guide tube and brain tissue to prevent reflux.

Optionally, an outer surface of the guide tube comprises a lubricious coating, for example of Parylene or PTFE. This improves the ease with which the guide tube can be inserted into the brain, reducing shear forces on the brain tissue and trauma to the tissue when the guide tube is passed into the brain.

Optionally an extreme distal end of the guide tube is rounded, bullet-shaped, or conical. This reduces coring of the brain tissue and trauma during insertion of the guide tube.

Optionally, the distal end of the guide tube has a diameter of at most 2.5 mm, preferably 1.8 mm, more preferably at most 1.6 mm, most preferably at most 1.3 mm. This range of dimensions is particularly suited to permit effective infusion while reducing trauma to the tissue.

Optionally, the through-bore of the guide tube has a diameter of between 0.2 mm and 1 mm, preferably between 0.4 mm and 0.7 mm, more preferably between 0.5 mm and 0.6 mm. This allows a fine cannula to be passed though it and into the tissues that has the appropriate dimensions to achieve effective tissue infusions.

Optionally, the guide tube is a rigid guide tube comprising a rigid material such as ceramic, for example zirconia ceramic; metal, for example titanium; or rigid plastic, for example polyether ether ketone.

These materials are biocompatible, will not cause significant image artefact on MRI and have properties suited to the requirements on the rigidity of the guide tube. This prevents the guide tube deflecting or distorting during its insertion, thereby ensuring it correctly reaches the target.

Optionally, the rigid guide tube has a length of between 50 mm and 300 mm, preferably between 100 mm and 250 mm. This allows the guide tube to reach common targets within the human brain, for example when held by an external apparatus such as a stereoguide or robot guide.

Optionally the rigid guide tube comprises a proximal end configured to be guided and/or held by a stereoguide or robot guide during insertion of the guide tube into the brain and/or during delivery of an infusate into the brain using the guide tube. This allows the guide tube to be inserted directly into the brain to the desired target location, for example during an acute procedure.

Optionally, a proximal end of the rigid guide tube has a larger diameter than the distal end of the guide tube. This allows the guide tube to be held more conveniently and securely by the stereoguide or robot guide. Optionally, the proximal end of the guide tube has a diameter of at most 7 mm, preferably at most 6 mm, more preferably at most 5 mm.

Optionally, the guide tube is a cuttable guide tube wherein a material and/or thickness of the guide tube is such that the guide tube can be cut by hand using a knife. This allows the guide tube to be easily adapted to the correct length during a procedure and before insertion.

Optionally, the cuttable guide tube has a length of between 25 mm and 150 mm. Preferably the maximum length of the cuttable guide tube is 110 mm. For example, the length may be between 50 mm and 110 mm. This allows the guide tube to reach common target volumes within the human brain, for example when its proximal end is secured to the skull.

Optionally, the cuttable guide tube is a multi-layered tube with inner and outer layers having different properties, for example where the layers are made of plastic.

Optionally, the cuttable guide tube comprises a porous layer to allow passage of air, the porous layer comprising a hydrophobic material. The porous layer is preferably in fluid communication with the through-bore of the guide tube. This allows for venting of air when a fluid transfer tube is passed into the guide tube, reducing damage to the tissue from compression of the air. A hydrophobic material ensures the porous material is not obstructed by liquid, so that its venting capability is preserved. Optionally, the hydrophobic material is superhydrophobic. Optionally, the porous layer comprises at least one of sintered PTFE, sintered polyurethane, ePTFE, silicone foam, polyurethane foam, shape memory polymer, microporous hollow extruded-polymer fibres, or electrospun polymer. Optionally, the microporous hollow extruded-polymer fibres and/or the electrospun polymer comprises at least one of polytetrafluoroethylene, polyvinylidene difluoride, polyurethane, polypropylene, and copolymers thereof.

Optionally, the porous layer may comprise a non-homogeneous material having varying stiffness and/or a plurality of materials having different stiffnesses. This is advantageous if the porous layer of the guide tube is constructed of a material that has insufficient column strength to maintain its length when inserted into brain tissue over a probe. The varying stiffness or plurality of different stiffnesses allows for a stiffer layer to provide support to the more compliant porous material.

Optionally, the stiffness of the guide tube is greater at or near the through-bore. This may help to support a fluid transfer tube passed into the guide tube.

Optionally, the porous layer comprises at least an inner layer and an outer layer, wherein the inner layer has greater stiffness than the outer layer and the inner layer is porous to air. Making the stiffer inner layer also permeable to air allows for improved venting of air from the throughbore of the guide tube.

Optionally, the inner layer comprises a plurality of holes through the inner layer. This is a convenient way to provide porosity without comprising the rigidity of the stiffer layer.

Optionally the inner layer is an innermost layer of the guide tube and provides the surface of the through-bore. This may help to support a cannula passed into the guide tube.

Optionally, the inner layer comprises at least one of: polyether ether ketone; nylon; polyurethane; polyester; a fluoropolymer such as polytetrafluoroethylene; a polymeric perfluoroether such as perfluoroalkoxy alkane, polyvinylidene difluoride, or fluorinated ethylene propylene; liquid crystal polymers; and mixtures or copolymers thereof. These materials have appropriate properties for the inner layer, for example in terms of rigidity.

Optionally, the inner layer is manufactured by a process comprising at least one of: sintering, extrusion with particulate leaching, micro-perforating a tube by drilling or by laser; weaving, braiding or electrospinning polymer fibres about a cylindrical former to form a tube of porous polymer material; and 3D printing a polymer in a porous form. These manufacturing methods are suitable for forming the narrow diameter structure required for the guide tube inner layer.

Optionally, the porous layer is an outermost layer of the guide tube. This is easier to manufacture, for example if the guide tube is formed from a sintered polymer or by depositing the porous layer onto a stiffer inner layer. This can also improve biointegration of the guide tube in chronic implantation, due to penetration of tissue into the pores of the porous outer layer.

Optionally, the guide tube further comprises a fluid-impermeable covering layer provided over the porous layer. Optionally, the covering layer is lubricious. Optionally, the covering layer comprises a heat shrink tube, for example comprising a polymer that is lubricious and impermeable to fluid. Suitable heat shrink polymers include Fluorinated Ethylene Propylene (FEP), Polyethylene Terephthalate (PET), Polyolefin and Polytetrafluoroethylene (PTFE).

Optionally, the reduced pressure regions may be provided by the covering layer. For example, the covering layer may comprise a heat shrink tube with a series of annular thickenings or groves along its axis that is applied over the porous layer.

Optionally, the guide tube is flexible. This can allow the guide tube to accommodate movement of the brain relative to the skull, particularly in chronic implantation of the guide tube.

Optionally, at least a proximal portion of the guide tube is resiliently deformable along an axial direction. This allows the guide tube to accommodate movement of the brain within the skull without significant movement of the distal end of the guide tube that might displace it from its intended target.

Optionally, the guide tube comprises at least an inner layer and an outer layer, wherein the inner layer has greater stiffness than the outer layer and the outer layer is resiliently deformable; and the inner layer does not extend into the proximal portion of the guide tube. By providing a region where the inner layer is absent, the outer layer in that region can deform to accommodate movement of the brain relative to the skull.

Optionally, the guide tube comprises at least an inner layer and an outer layer, wherein the inner layer has greater stiffness than the outer layer and the outer layer is resiliently deformable; and in the proximal portion of the guide tube, the inner layer is configured to provide a spring. A spring configuration provides continuity of the inner layer for greater stability, while still allowing for relative movement of the brain and skull to be accommodated.

Optionally, a proximal end of the guide tube comprises an enlargement configured for securing in a burr hole in a skull. This allows for forming a seal between the guide tube and the skull to prevent fluid leakage out of the brain and ingress of material into the brain.

Optionally, the enlargement comprises an increased-diameter portion configured to engage with a guide hub for securing to the skull. This allows for secure engagement of and sealing between the guide tube and guide hub.

Optionally the increased diameter portion is an over-mould onto the stiffer inner layer. For example, the increased diameter portion may be a PEEK over-mould onto a perforated PEEK inner tube or a polyurethane over-mould onto an air porous polyurethane inner tube.

Optionally, engagement of the increased-diameter portion of the guide tube with the guide hub forms a fluid seal between the guide tube and the guide hub. This prevents cerebrospinal fluid and infusate that may be introduced via the guide tube, leaking out, and also prevents ingress of unwanted material into the skull.

Optionally, the guide tube comprises a resiliently deformable outer layer, and engagement of the increased-diameter portion of the guide tube with the guide hub comprises compression of the outer layer. This reduces the need for other sealing means such as o-rings which may reduce the size and complexity of the apparatus.

Optionally, the through-bore of the guide tube within the increased-diameter portion increases in diameter towards the proximal end of the increased-diameter portion. This assists in inserting a fluid transfer tube such as a cannula into the guide tube by guiding the tube into the through-bore.

Optionally, the increased-diameter portion comprises a non-compliant core located inward of the outer layer. This provides a secure proximal portion for insertion of the guide tube and engagement with the guide hub.

Optionally, the non-compliant core is configured to allow the passage of gas through the non-compliant core. This allows the non-compliant core to enhance the gas venting capability of the guide tube by increasing the surface area through which gas can be vented. Optionally, the non-compliant core comprises an air porous material such as sintered polyurethane or PEEK. This will assist in the venting of air when the fluid transfer tube is inserted down the bore of the guide tube. Alternatively or additionally the non-compliant core comprises air venting channels through it to assist in venting air from the porous layer of the guide tube to the atmosphere.

Optionally, the non-compliant core is located inward of the porous layer. This allows the porous layer to vent gas around the non-compliant core.

Optionally, the non-compliant core is located outward of the inner layer. This allows the non-compliant core to be secured to the stiffer inner layer without affecting the shape and position of the inner layer in the proximal region.

Optionally, the inner layer does not extend to an extreme proximal end of the increased-diameter portion, optionally wherein the inner layer extends through at most 80%, preferably at most 60% of the increased-diameter portion. This allows the properties of the increased-diameter portion to be more completely determined by the non-compliant core. It also exposes more of the non-compliant core to the through-bore in the increased-diameter portion, which may be advantageous especially where the non-compliant core is configured to vent gas.

According to a second aspect, there is provided a neurosurgical apparatus comprising: the guide tube comprising an increased-diameter portion configured to engage with a guide hub for securing to the skull; and a guide hub for securing to the skull of a patient before insertion of the guide tube into the brain, the guide hub having a passage for the guide tube therethrough. The guide hub provides a fixed point on the skull into which the guide tube can be inserted and secured. This may be useful for patients requiring regular procedures, as the guide hub and optionally the guide tube can be left in place between infusion procedures.

Optionally, the apparatus further comprises a fluid transfer tube, preferably a cannula, configured for insertion into the through-bore of the guide tube. The fluid tube can be used for introducing an infusate and can be easily connected to an external pump or syringe.

According to a third aspect, there is provided a neurosurgical apparatus comprising: the guide tube of the first aspect; and a fluid transfer tube, preferably a cannula, configured for insertion into the through-bore of the guide tube. Providing the guide tube and fluid transfer tube together is convenient since it ensures they can be made mutually compatible for any desired procedure. The fluid tube can be used for introducing an infusate and can be easily connected to an external pump or syringe.

Optionally, the fluid transfer tube comprises a depth-controlling stop configured to engage with a proximal end of the guide tube to form a fluid seal between the fluid transfer tube and the guide tube, optionally wherein the depth-controlling stop engages with an increased-diameter portion of the proximal end of the guide tube. This ensures that the fluid transfer tube is inserted to the correct depth, so that fluid reaches the correct region of the brain.

Optionally, when the guide tube comprises a rigid material or the guide tube comprises a proximal end configured to be guided and/or held by a stereoguide or robot guide during insertion of the guide tube into the brain, the fluid transfer tube may comprise a rigid material, for example fused silica. Optionally, when the guide tube is cuttable and/or flexible the fluid transfer tube may be flexible. This matches the properties of the fluid transfer tube to the guide tube to ensure they both handle similarly and correctly during insertion and use.

Optionally, the apparatus of the second or third aspects further comprises a probe configured for insertion into brain tissue, the probe comprising: a rod having a distal end that is rounded or conical in shape; and a spike extending axially from the distal end of the rod, the spike having a narrower diameter than the rod and having an extreme distal end configured for dissecting brain tissue. This probe can be used for forming a track in the brain prior to insertion of the guide tube.

Optionally, the apparatus of the second or third aspects further comprises a delivery probe configured for insertion into the through-bore of the guide tube, wherein a distal end of the delivery probe is configured for dissecting brain tissue. The delivery probe allows the guide tube to be delivered easily to the target position, particularly where the guide tube is flexible and may deform when inserted into the brain without using a delivery probe.

According to a fourth aspect, there is provided a neurosurgical apparatus comprising: the guide tube of the first aspect; and a probe configured for insertion into brain tissue, the probe comprising: a rod having a distal end that is rounded or conical in shape; and a spike extending axially from the distal end of the rod, the spike having a narrower diameter than the rod and having an extreme distal end configured for dissecting brain tissue. This apparatus can be used to form a track in the brain prior to insertion of the guide tube using the probe, and then insert the guide tube.

According to a fifth aspect, there is provided a neurosurgical apparatus comprising: the guide tube of the first aspect; and a delivery probe configured for insertion into the through-bore of the guide tube, wherein a distal end of the delivery probe is configured for dissecting brain tissue. The delivery probe allows the guide tube to be delivered easily to the target position, particularly where the guide tube is flexible and may deform when inserted into the brain without using a delivery probe.

According to a sixth aspect, there is provided a method for implantation of a guide tube for convection-enhanced delivery of an infusate to brain parenchyma, the method comprising: inserting a guide tube into the brain until a distal end of the guide tube reaches a planned location in the brain, wherein the guide tube comprises a through-bore for passage of a fluid transfer tube and a distal end comprising a sealing region; compressing brain tissue adjacent to said sealing region to form a reflux-inhibiting seal around the distal end of the guide tube; forming a reduced-pressure region in which the compression of the brain tissue is lower than the compression of brain tissue in both a proximally adjacent region and a distally adjacent region to the reduced-pressure region.

Using the guide tube as described allows implantation of a system which will have reduced reflux of fluid introduced through the fluid transfer tube due to the reduced-pressure region provided by the guide tube.

Optionally, the method further comprises, prior to inserting the guide tube into the brain, inserting a probe into the brain parenchyma to form a guide tube track extending to the planned location, wherein: the guide tube has a diameter at least that of the probe; and inserting the guide tube into the brain comprises inserting the guide tube into the guide tube track. Forming a track first using a probe in this manner can reduce trauma to the brain tissue caused by advancing the guide tube into the brain.

Optionally, the probe comprises: a rod having a distal end that is rounded or conical in shape; and a spike extending axially from the distal end of the rod, the spike having a narrower diameter than the rod and having an extreme distal end configured for dissecting brain tissue. The spike allows for the opening of the track with reduced trauma to brain tissue.

Optionally, the method further comprises cutting the guide tube to a desired insertion length with respect to a fixation point in the skull prior to insertion of the guide tube into the brain. This means that the guide tube is provided at exactly the required length based in in-situ measurements, rather than relying on prior estimates.

According to a seventh aspect, there is provided a method of convection-enhanced delivery of an infusate to brain parenchyma comprising: implanting a guide tube in the brain using the method of the sixth aspect; advancing a delivery probe through the through-bore along the axis of the guide tube to form a fluid transfer tube track extending from the distal end of the guide tube; passing a fluid transfer tube through the through-bore of the guide tube and into the brain along the fluid transfer tube track; and delivering the infusate into the brain via the fluid transfer tube.

Optionally, the inserting of the guide tube is carried out with the delivery probe within the through-bore, such that a distal end of the delivery probe extends to or just beyond the distal end of the guide tube. This can help to maintain the shape and rigidity of the guide tube during insertion.

Optionally, the fluid transfer tube track has a narrower diameter than the guide tube track. This provides a step at the distal end of the guide tube, which will contribute to improved sealing and reduced reflux.

According to an eighth aspect, there is provided a method of convection-enhanced delivery of an infusate to brain parenchyma comprising: implanting a guide tube in the brain using the method of the sixth aspect, wherein inserting the guide tube into the brain comprises inserting the guide tube with a fluid transfer tube within the through-bore, preferably wherein a positive pressure of the infusate is provided within the fluid transfer tube during insertion of the guide tube; and delivering the infusate into the brain via the fluid transfer tube.

### LIST OF FIGURES

Embodiments of the present invention will now be described by way of non-limitative example with reference to the accompanying drawings, in which:

FIG. 1 is a close-up of the distal end of a guide tube and cannula;

FIG. 2 is a cross-section view of the distal end of the guide tube;

FIG. 3 shows a fluid transfer tube or cannula;

FIG. 4 shows an insertion guide for holding a guide tube for use with a stereoguide;

FIG. 5 is a cross-section of a guide tube with a sealing region at its distal end for use with the insertion guide of FIG. 4, assembled with a cannula in its through-bore;

FIG. 6 is a close-up cross-sectional view of a section of the sealing region; and

FIG. 7 is a cross-section of a guide tube with a sealing region at its distal end, and which comprises a perforated inner layer with a spiral-cut proximal portion;

FIG. 8 shows the inner layer of the guide tube of FIG. 7;

FIG. 9 is a cross-section of a guide tube with a sealing region at its distal end, and which comprises a perforated inner layer;

FIG. 10 shows the inner layer of the guide tube of FIG. 9;

FIG. 11 shows the guide tubes of FIGS. 8 and 10 with the outer layer;

FIG. 12 shows a guide hub for securing to the skull;

FIG. 13 is a cross-sectional view of the guide hub of FIG. 12;

FIG. 14 is a cross-sectional view of an assembled neurosurgical apparatus;

FIG. 15 shows the assembled neurosurgical apparatus of FIG. 14;

FIG. 16 shows a bubble vent attached to the proximal end of the fluid transfer tube;

FIG. 17 shows an exploded view of the bubble vent;

FIG. 18 shows an alternative design of bubble vent;

FIG. 19 shows an exploded view of the bubble vent of FIG. 18;

FIG. 20 shows a cross section of the bubble vent of FIGS. 18 and 19;

FIG. 21 shows a close up view of the bubble vent of FIG. 20 in use; and

FIG. 22 shows a probe used to form a track in the brain into which the guide tube can be inserted.

### DETAILED DESCRIPTION

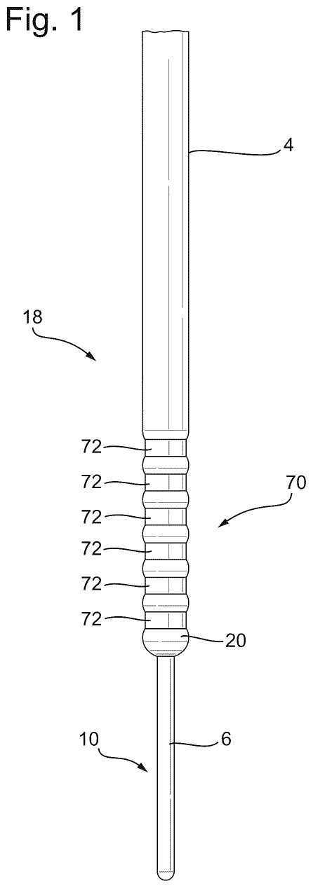

To address the limitations of prior art guide tubes as described above, the present disclosure provides a guide tube 4 such as that shown in FIG. 1. FIG. 1 shows the distal end 18 of a guide tube 4. The guide tube 4 is for use with a fluid transfer tube 6, such as shown in FIG. 3 and discussed in more detail below. The fluid transfer tube 6 (for example a cannula) is for providing fluid access to, for example to permit fluid transfer to or from, the brain of a mammal. The guide tube 4 is configured for insertion into the brain. The guide tube 4 and fluid transfer tube 6, optionally together with other components described below, may be referred to as a cannula assembly, neurosurgical cannula assembly, or neurosurgical apparatus.

The guide tube 4 is configured to be inserted into the brain coaxial with a trajectory to a target within the brain. The trajectory may be established with image guidance, and may be defined between an entry point on the skull and the target. The target may be a particular region of or location in the brain of the mammal.

The distal end 18 of the guide tube 4 may be inserted to a predetermined location in the brain along the trajectory to the target. The predetermined location may not be the therapeutic target point itself, but may be a point along the trajectory proximal of the target, since the fluid transfer tube 6 will generally extend beyond the distal end 18 of the guide tube 4. The proximal end 28 of the guide tube 4 may be guided by and held during use in a stereoguide or robot guide.

The distal end 18 of the guide tube 4 may have a diameter of at most 2.5 mm, preferably 1.8 mm, more preferably at most 1.6 mm, most preferably at most 1.3 mm. The guide tube 4 may comprise an increased diameter portion at its proximal end, as will be discussed further below. The maximum dimensions of the guide tube 4 discussed here generally exclude the increased diameter portion if present.

The guide tube 4 may comprise ceramic, for example zirconia ceramic, metal, for example titanium or stainless-steel, or plastic, for example polyether ether ketone. The preferred material may differ depending on the specific application, for example as discussed further below.

As shown in the close-up view of FIG. 1, an extreme distal end of the guide tube 4 may be rounded, bullet-shaped, or conical. This helps to reduce trauma to the brain tissue as the guide tube 4 is inserted.

An outer surface of the guide tube 4, in particular at the distal end 18, may be configured to make contact with brain tissue following insertion of the guide tube 4 into the brain. To improve tissue adherence, tissue integration capability, lubricity, or other desirable properties, at least an outer surface of the guide tube 4 may comprise a lubricious coating. For example, the outermost surface may be post processed with, for example, plasma treatment or have an appropriate coating applied to provide it with the desired properties. For example, a lubricious coating of Parylene or polytetrafluoroethylene (PTFE) may be used to reduce shear forces on brain tissue when the guide tube 4 is passed into the brain. Alternatively a lubricious heat shrink plastic tube may be applied to the outer surface of the guide tube. For example the heat shrink tube may be a formed of PTFE, FEP, PET, or Polyolefin.

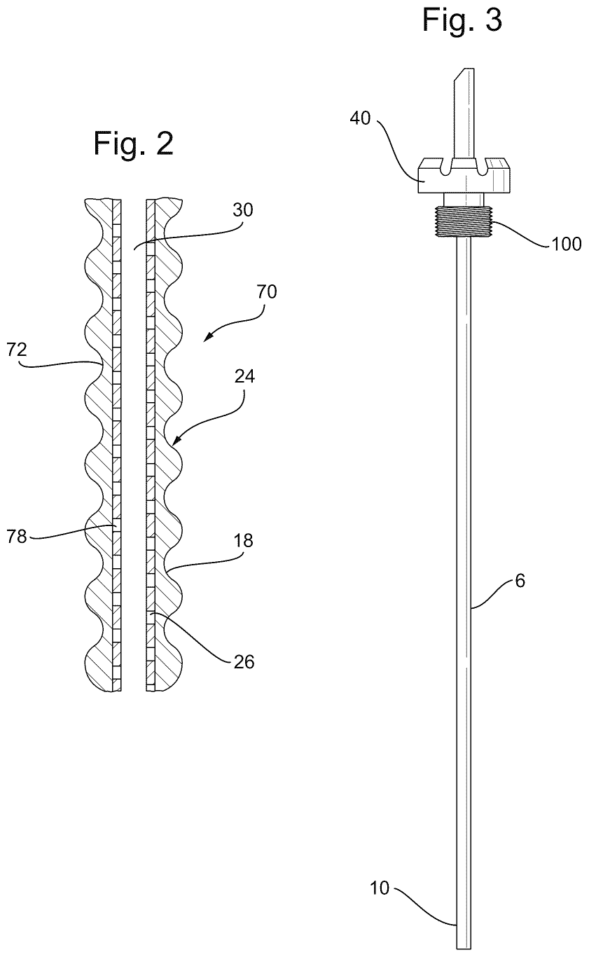

FIG. 2 shows a cross-sectional view of the distal end 18 of an exemplary guide tube 4. The guide tube 4 comprises a through-bore 30 for passage of the fluid transfer tube 6, such as a cannula. The through-bore 30 may have a diameter of between 0.2 mm and 1 mm, preferably between 0.4 mm and 0.7 mm, more preferably between 0.5 mm and 0.6 mm. Advantageously, the through-bore 30 of the guide tube 4 is sized to be a close fit to the outer surface of the fluid transfer tube 6. A close fit avoids providing a route for reflux of infusate out of the brain.

FIG. 3 shows an example of a fluid transfer tube 6. The fluid transfer tube 6 is configured to permit fluid transfer to or from the brain of a mammal. In particular, the fluid transfer tube 6 is used to transfer fluid to or from a target brain volume. The fluid transfer tube 6 is configured for insertion into the through-bore 30 of the guide tube 4 into the brain. The fluid transfer tube 6 is passed along the through-bore 30 from the proximal end 28 of the guide tube 4 to the distal end 18. The fluid transfer tube 6 typically projects beyond the guide tube's distal end 18 so that the distal end 10 of the fluid transfer tube 6 is positioned at the brain target.

The fluid transfer tube 6 may have an external diameter of between 0.4 mm and 0.7 mm, preferably between 0.5 mm and 0.6 mm, for example 0.5 mm. The fluid transfer tube 6 may have through-bore (or inner diameter) of between 0.1 mm and 0.4 mm. The length of the fluid transfer tube 6 may be at least 100 mm. In general, the fluid transfer tube 6 may have a length at least that of the guide tube 4.

The fluid transfer tube 6 may be formed from a rigid material, for example fused silica, a metal such as titanium or stainless steel or a stiff biocompatible plastic such as polyetheretherketone (PEEK). Alternatively, the fluid transfer tube 6 may be formed from a flexible material, for example a flexible biocompatible plastic. The choice of whether to use a rigid or flexible fluid transfer tube 6 may be made based on the specific application and the properties of the guide tube 4. Preferably, the distal end 10 of the fluid transfer tube 6 is rounded, conical, or bullet shaped to minimise tissue trauma upon its insertion, as described for the guide tube 4.

The fluid transfer tube 6 may have a fluid connector at its proximal end configured for connecting to a fluid infusion line or directly to a syringe and infusion pump. The fluid transfer tube 6, in particular a proximal end of the fluid transfer tube 6, may comprise a depth-controlling stop 40 configured to engage with a proximal end 28 of the guide tube 4 to form a fluid seal between the fluid transfer tube 6 and the guide tube 4. The depth-controlling stop 40 may engage with an increased-diameter portion of the proximal end 28 of the guide tube 4. Engagement of the distal face of the depth-controlling stop 40 on the fluid transfer tube 6 with the proximal face of an increased diameter portion of the guide tube 4 may form the fluid seal. The position of the depth-controlling stop 40 along the length of the fluid transfer tube 6 may be adjustable. This allows for the length of the fluid transfer tube 6 that protrudes beyond the distal end 18 of the guide tube 4 to be adjusted. Alternatively, the depth controlling stop may be fixed to the fluid transfer tube and the depth of insertion of the fluid transfer tube adjusted by cutting it to the desired length with respect to the stop using a knife.

In contrast to prior art devices, and as shown in FIG. 1 and FIG. 2, the distal end 18 of the guide tube 4 comprises a sealing region 70. The sealing region 70 is configured to compress surrounding brain tissue to form a reflux-inhibiting seal around the distal end 18 of the guide tube 4. The sealing region 70 comprises at least one reduced-pressure region 72 in which the compression of the brain tissue is lower than the compression of brain tissue in both a proximally adjacent local region and a distally adjacent local region. The proximally adjacent region and the distally adjacent region are adjacent with respect to the reduced-pressure region 72.

The guide tube 4 (or the cannula assembly where the fluid transfer tube 6 is inserted into the guide tube 4 prior to insertion into the brain) is preferably passed into the brain down a pre-made track as will be described in more detail below. As the guide tube 4 enters the track, the track is gently dilated up to the larger diameter of the guide tube 4. When liquid is driven down the fluid transfer tube 6 and into the brain through its distal end, the liquid will tend to follow the path of least resistance and pass back along the outside diameter of the fluid transfer tube 6 at its interface with the tissue. The refluxing liquid will travel back along the outside of the fluid transfer tube 6 towards the step 20 created by the change in diameter at the distal end 18 of the guide tube 4 from which the fluid transfer tube 6 extends. A high-pressure zone in the tissue just distal to the step 20 created by insertion of the guide tube 4 will compress the tissue interface and resist reflux. The rising liquid pressure will then drive the fluid radially into the tissues.

However, as mentioned above in relation to prior art devices, this process of convection will continue until the infused liquid pressure overwhelms the tissue pressure at the step 20. Once the tissue pressure is overwhelmed, the liquid will overwhelm the step 20 and reflux up the outside of the guide tub 4 and away from the target region, which is undesirable. The provision of the sealing region 70 (for example using a series of circumferential grooves along the outside diameter of the guide tube 4 in FIGS. 1 and 2) will, in effect, create a series of ring seals along the outside of the guide tube 4 that will provide cumulative resistance to liquid refluxing back along the length of the guide tube 4. This will provide reflux resistance with a smaller outside diameter of the guide tube 4, since the cumulative resistance is overall greater than the resistance created by a single large step. This in turn means the guide tube 4 is less traumatic to the brain tissue than currently available reflux resistant cannulas and guide tubes, as well as being more effective in reducing reflux.

The sealing region 70 may comprise one or a plurality of reduced-pressure regions 72. For example, the guide tube 4 shown in FIG. 1 has a sealing region 70 comprising six reduced-pressure regions 72. Where the sealing region 70 comprises a plurality of reduced-pressure regions 72, the proximally adjacent region and/or the distally adjacent region (in which the compression of brain tissue is higher than in the reduced-pressure region 72) may be shared between adjacent reduced-pressure regions 72. In other words, the proximally adjacent region of a first reduced-pressure region 72 may be also be the distally adjacent region of a second reduced-pressure region 72. FIG. 6 shows a further close-up view of part of the sealing region 70. The plurality of reduced-pressure regions 72 may be equally-spaced along a length of the guide tube 4. A spacing between the reduced-pressure regions 72 may be at most 2 mm, preferably at most 1.5 mm, more preferably at most 1 mm. The spacing between the reduced-pressure regions 72 may be at least 0.5 mm. For example, the spacing may be 1 mm apart. A width W2 of each pressure-reduced region 72 may be at least 0.5 mm, optionally at least 1 mm. The width of each pressure-reduced region 72 may be at most 2 mm, optionally at most 1.5 mm.

The reduced-pressure regions 72 may be provided over at least 5% and/or over at least 2 mm of a length of the guide tube 4. That is, a distance between the most distal reduced-pressure region 72 and the most proximal reduced-pressure region 72 may be at least 5% of a length of the guide tube 4, and/or at least 2 mm. Preferably, the reduced-pressure regions 72 may be provided over at least 10% and/or at least 4 mm of the length of the guide tube 4. In some situations it may be advantageous to provide the reduced-pressure regions 72 over substantially all of the length of the guide tube 4, excluding the increased diameter portion 90 if present. For example, at least the entire distal portion 18 of the guide tube 4 may be provided with reduced-pressure regions 72. All but the most proximal 2 cm (not including the increased diameter portion 90 if present), optionally 5 cm, optionally 10 cm of the guide tube 4 may be provided with reduced-pressure regions 72.

To be most effective in suppressing reflux, the reduced-pressure regions 72 are preferably close to the extreme distal end of the guide tube 4. At least one of the reduced-pressure regions 72 may be at most 20 mm, optionally 10 mm, optionally 5 mm, further optionally 2 mm away from the extreme distal end of the guide tube 4. At least one of the reduced-pressure regions 72 may be at most 25%, optionally 15%, optionally 10%, further optionally 5% of a length of the guide tube 4 away from the extreme distal end of the guide tube 4.

The reduced-pressure region or regions 72 may be provided in any suitable way. The sealing region 70 may comprise one or a plurality of features on an outer diameter of the guide tube 4 configured to provide the reduced-pressure region or regions 72. The feature(s) may comprise a change in the outer diameter of the guide tube 4, such that the outer diameter of the guide tube 4 varies in the sealing region 70. For example, in the guide tube of FIG. 1, the reduced-pressure region(s) 72 are provided by grooves in an outer surface of the guide tube 4. That is, the outer diameter of the guide tube 4 is reduced in the reduced-pressure region(s) 72 compared to the outer diameter of the guide tube 4 in the remainder of the distal region 18 outside of the sealing region 70. Alternatively, or additionally, the reduced-pressure region(s) 72 may be provided between protrusions 94 from an outer surface of the guide tube 4 as shown in FIG. 6. That is, the outer diameter of the guide tube 4 may be increased in the proximally adjacent region and distally adjacent region either side of the reduced-pressure region 72 compared to the outer diameter of the guide tube 4 in the remainder of the distal region 18 outside of the sealing region 70.

The reduced-pressure region(s) 72 may also be provided by a combination of protrusions and grooves. Thus, the reduced-pressure region(s) 72 may have a diameter smaller than the guide tube outer diameter, and there may be regions either side of the reduced-pressure region(s) 72 that have a diameter larger than the guide tube diameter.

The groove may have a depth of at most 0.5 mm, or the protrusions 94 may have a height T2 of at most 0.5 mm. The groove may have a depth of at least 0.1 mm, or the protrusions 94 may have a height T2 of at least 0.1 mm. Preferably, the groove may have a depth of 0.2 mm or the protrusions 94 may have a height T2 of 0.2 mm.

The groove or protrusions 94 may extend around the entire circumference of the guide tube 4. This ensures that the reduced-pressure region(s) 72 extend(s) around the entire circumference of the guide tube 4, thereby improving the sealing effect of the sealing region 70.

The guide tube 4 may be a rigid guide tube that is inserted to a predetermined location in the brain along a trajectory that is established with image guidance and whose proximal end is guided by and held during use by a stereoguide or robot guide. Alternatively, the guide tube 4 may be a cuttable guide tube, that is cut to a prescribed length and whose distal end is inserted to a predetermined location in the brain with image guidance and whose proximal end is fixed in the skull during use. These embodiments will be described in detail hereinafter.

The guide tube 4 may comprise a proximal end configured to be guided and/or held by a stereoguide or robot guide during insertion of the guide tube 4 into the brain and/or during delivery of an infusate into the brain using the guide tube 4. The guide tube 4 may comprise an adjustable clamp 92 that is used to hold the guide tube 4 at the correct insertion depth in the stereoguide. The clamp 92 is adjustable to allow the guide tube 4 to be set for different depths of insertion depending on the target region in the brain and the particular stereoguide setup being used.

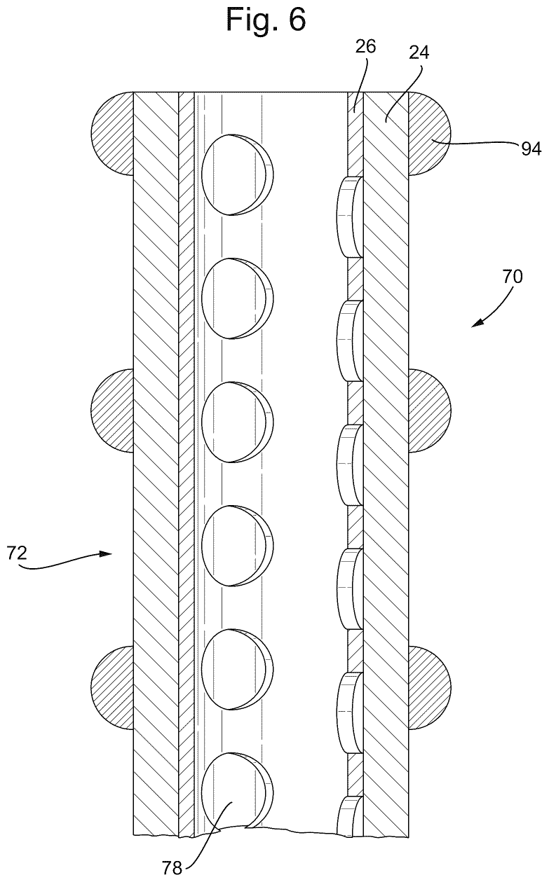

As shown in FIG. 5, the proximal end 28 of the guide tube 4 may have a larger diameter than the distal end 18 of the guide tube 4. The proximal end 28 of the guide tube 4 may have a diameter of at most 7 mm, preferably at most 6 mm, more preferably at most 5 mm. This larger diameter at the proximal end 28 can allow the guide tube to be held or located more securely in the stereoguide or robot guide. In this case, the larger-diameter proximal end 28 is not inserted into the brain during use. Only the smaller diameter distal end 18 enters the brain during use.

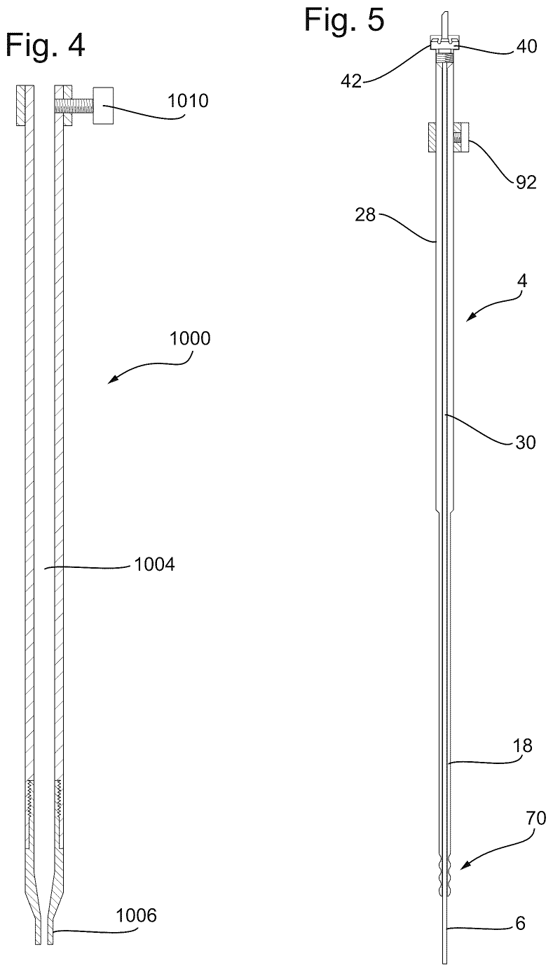

The guide tube 4 may be held in the stereoguide or robot guide using an insertion guide 1000 such as that shown in FIG. 4. The insertion guide 1000 has an outer diameter suitable for holding in the stereoguide or robot guide. The insertion guide has a through-bore 1004 having a diameter approximately equal to the outer diameter of the proximal end 28 of the guide tube 4. The diameter of the through-bore 1004 should be slightly larger than the diameter of the proximal end 28 of the guide tube 4, so that the guide tube 4 can be easily inserted and moved up and down in the insertion guide 1000, but not so much larger that significant lateral movement of the guide tube 4 in the insertion guide 1000 is possible. The insertion guide 1000 may have a clamp 1010 to hold the guide tube 4 in place once the guide tube 4 has been fully inserted. The clamp 1010 ensures the guide tube 4 does not move while fluid is being delivered into the brain. The insertion guide 1000 may narrow at its distal end 1006 so that the diameter of the through-bore at the distal end 1006 is approximately equal to the diameter of the distal end 18 of the guide tube 4.

The guide tube 4 shown in FIG. 4 is a rigid guide tube. The guide tube 4 of FIG. 4 and FIG. 5 comprises a rigid material, for example ceramic, a metal such as titanium or stainless-steel, or a rigid plastic material such as PEEK. When the guide tube 4 is rigid, it can be inserted directly into the brain without the need to form a track in the brain beforehand, or use a delivery probe to support the guide tube 4 as it is inserted into the brain. A rigid guide tube 4 may be particularly suited for acute treatments, where the guide tube 4 is inserted into the brain to perform a particular procedure and then removed once the procedure is complete. This type of guide tube 4 is generally used with the stereoguide or robot guide. The rigid guide tube may have a length of between 50 mm and 300 mm, preferably between 100 mm and 250 mm. This allows it to extend from where it is held by the stereoguide or robot guide to the target volume in the brain.

When a rigid guide tube 4 is used, the fluid transfer tube 6 may be inserted into the through-bore 30 of the guide tube 4 before the guide tube 4 is inserted into the brain, so that the guide tube 4 and fluid transfer tube 6 are inserted into the brain as a single unit. In this case, in use the fluid transfer tube 6 is primed with a liquid prior to insertion of the cannula assembly into the brain tissue. This prevents air being trapped in the fluid transfer tube 6 that would then be forced out into the brain when liquid is passed into the fluid transfer tube 6. The fluid transfer tube 6 may be positioned and retained within the through-bore 30 by a releasable compression seal 42 at the proximal end of the guide tube 4. The compression seal may interact with the depth-controlling stop 40 on the fluid transfer tube 6, if present. The fluid transfer tube 6 may also be rigid. For example, the fluid transfer tube 6 may comprise or be formed from a rigid material such as fused silica, a metal such as titanium or stainless steel, or a stiff biocompatible plastic such as polyetheretherketone (PEEK).

The fluid transfer tube 6, when inserted through the through-bore 30 of the guide tube 4 prior to its insertion in the brain, may be positioned so that its distal end 10 is at least aligned with the distal end 18 of the guide tube 4 or projects beyond it. The guide tube 4 may be inserted into the brain tissue with the fluid transfer tube 6 projecting beyond its distal end by a predetermined length so that when the distal end 18 of the guide tube 4 reaches the target region, the distal end 10 of the fluid transfer tube 6 will also reach its planned target. Alternatively, after insertion of the guide tube 4 the distal end 10 of the fluid transfer tube 6 may be advanced through the tissue to the selected target.

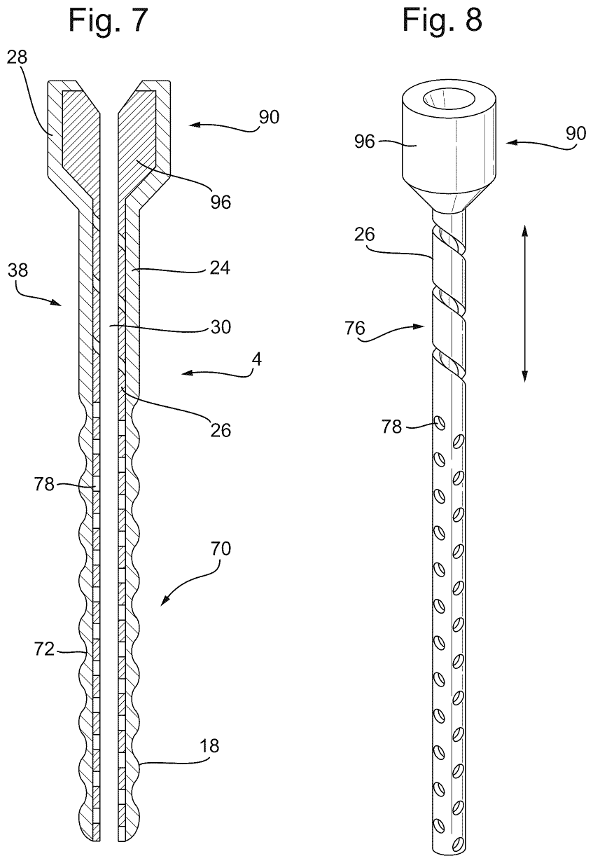

FIGS. 7-15 illustrate an alternative guide tube design to that of FIGS. 4 and 5 in which the guide tube 4 is secured to the skull following insertion and during the delivery of an infusate using the guide tube 4. In contrast to use of the guide tube in the embodiment held by a stereoguide during the delivery of an infusate, wherein only a single target infusion can be accomplished at a time, use of the embodiment with bone fixation facilitates the infusion through several implanted devices simultaneously. This greatly reduces procedure time and the risk to patients. This type of guide tube 4 can also be left in place for extended periods of time for use when chronic, or chronic intermittent infusions are required.

A material and/or thickness of the guide tube 4 may be such that the guide tube 4 can be cut by hand using a knife or sharp blade. The guide tube 4 may be cut before insertion to the desired insertion length with respect to the fixation of its proximal end 28 at the skull. The guide tube 4 may be cut with a sharp blade in a cutting jig. The guide tube 4 may be cut with an indwelling plastic stylet inserted within the through-bore 30 of the guide tube 4. The stylet is cut with the guide tube 4 and supports the guide tube 4 during the cutting process. Preferably the guide tube 4 is cut in the same cross-sectional plane as one of the reduced-pressure regions 72, particularly where the reduced-pressure regions are provided by circumferential grooves. This will provide the distal end 18 of the guide tube 4 with a relatively rounded profile which will cause less trauma to the tissues upon its insertion than an orthogonal cut through the largest diameter of the guide tube 4.

The cuttable guide tube may have a length of between 25 mm and 150 mm. Preferably the maximum length of the cuttable guide tube is 110 mm. For example, the length may be between 50 mm and 110 mm. This allows the guide tube to reach common target volumes within the human brain, for example when its proximal end is secured to the skull.

FIG. 7 shows a cross-section of an example of this type of guide tube 4. As for the guide tube 4 of FIG. 4, the guide tube 4 comprises a proximal end 28, a distal end 18, and a through-bore 30 for passage of a fluid transfer tube 6 (such as a cannula). The guide tube 4 has a sealing region 70 comprising one or a plurality of reduced-pressure regions 72 in the form of circumferential grooves disposed along its outer diameter.

In use the guide tube 4 has a proximal end 28 that does not enter the brain but is fixed at or near the skull of a patient so as to secure the position of the guide tube 4 in the brain. The guide tube 4 of the invention may be provided with an enlargement at the proximal end 28 sized and shaped for securing in a burr hole in a skull as with prior art arrangements. The enlargement may comprise an increased-diameter portion 90. For example, the guide tube 4 may comprise an inner layer 26 overlaid with an outer layer 24 as shown in FIG. 2 and described further below. The inner layer 26 may have an over-moulded increased diameter portion 90 on its proximal end to create a flare or stop to limit its depth of insertion and facilitate its fixation in the skull.

Advantageously, the guide tube 4 may be inserted through a burr hole in the skull that has already been fitted with a guide hub 50 such as shown in FIG. 12 and FIG. 13. The guide hub 50 is for securing to the skull of a patient before insertion of the guide tube 4 into the brain. The separate guide hub 50 may be provided as part of a neurosurgical apparatus comprising the guide tube 4 and optionally the fluid transfer tube 6 and/or other components discussed below. The guide hub 50 has a passage 52 for the guide tube 4 therethrough. The guide hub 50 provides a fixed datum point at the skull, from which the probes, guide tube 4, and fluid transfer tube direction and length can be directed.

Where a guide hub 50 is employed, the guide tube 4 may have an increased diameter portion 90 such as described above, open at its proximal end 28 for securing in the guide hub 50 in use. The increased diameter portion 90 of the guide tube 4 may be configured to engage with the guide hub 50, for example by engaging with a corresponding shaped seat in the guide hub passage 52. The increased diameter portion 90 may be cylindrical, part spherical or conical (‘flared’), for example.

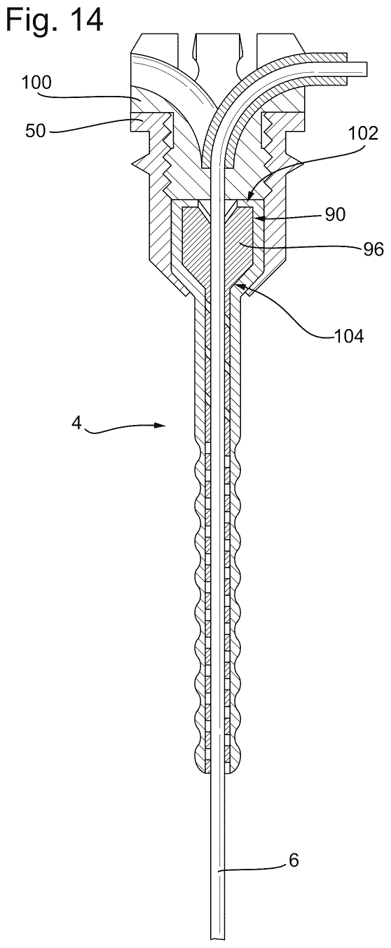

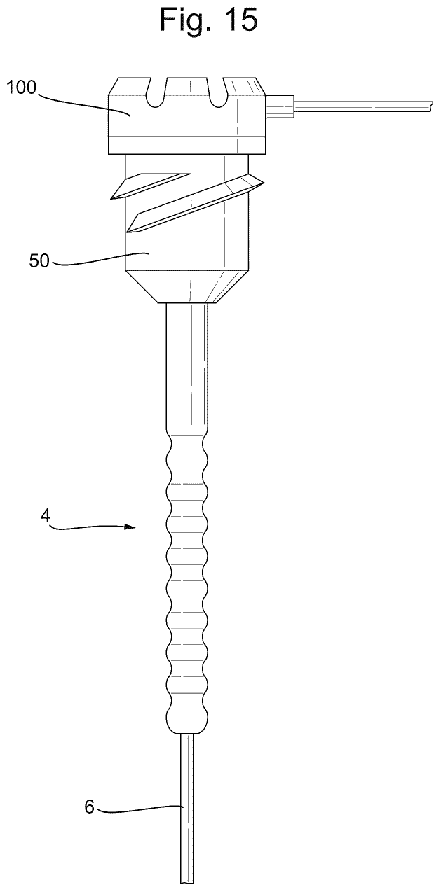

The proximal end 28 of the guide tube 4 can then be secured to the guide hub 50, for example by a fitting such as a screw fitting 100 with a bore therethrough. As shown in FIG. 14, the screw fitting 100 screws down onto the proximal face 102 of the increased diameter portion 90 of the guide tube 4, compressing it onto the corresponding shaped seat 104 in the guide hub passage 52. The assembled neurosurgical apparatus is also shown in FIG. 15.

Advantageously the compression of the increased diameter portion 90 of the guide tube 4 may form a fluid seal between the guide tube 4 and the guide hub 50, and between the guide tube 4 and the screw fitting 100. Interposition of a deformable washer, such as a silicone washer, between the distal end of the screw fitting 100 and the proximal face 102 of the increased diameter portion 90 of the guide tube 4 may radially compress and seal around the fluid transfer tube 6. This may also fix the position of the fluid transfer tube 6 with respect to the guide hub 50 and thereby the skull. In compressing around the fluid transfer tube 6, such a washer can also create a fluid seal between the fluid transfer tube 6 and the guide tube 4.

A fluid seal may also be created between the fluid transfer tube 6 and guide tube 4 if the fluid transfer tube 6 has a depth-controlling stop 40 at a proximal end. The depth-controlling stop 40 may be a threaded stop which is screwed into a complimentary thread within the bore of the guide hub 50. For example, the depth-controlling stop 40 may create a conical seal between a conical form on the distal end of the depth-controlling stop 40 and the increased diameter portion 90 of the guide tube 4.

At least the outer layer 24 of the guide tube 4 may be resiliently deformable, as will be discussed further below. In this case, when the increased-diameter portion 90 is inserted into the guide hub 50 and the distal face of the depth-controlling stop 40 on the fluid transfer tube 6 is brought into positive engagement with the increased-diameter portion 90, the deformable outer layer 24 is compressed against the increased-diameter portion 90 and forms a fluid seal. This seals a potential leak path of liquid that could otherwise reflux between the through-bore 30 of the guide tube 4 and the fluid transfer tube 6 to the outside of the CNS. The seal also closes a path of potential bacterial ingress into the brain.

Engagement of the increased-diameter portion 90 of the guide tube 4 with the guide hub 50 may form a fluid seal between the guide tube 4 and the guide hub 50. For example, this could be achieved through engagement of a distal face of the increased diameter portion 90 with a corresponding shaped seat in the guide hub passage 52. The force compressing the proximal face 102 of the increased diameter portion 90 of the guide tube 4 is also transmitted to the distal face of the increased diameter portion 90 that is in contact with the correspondingly shaped seat in the guide hub 50. Where the guide tube 4 comprises a resiliently deformable outer layer 24, engagement of the increased-diameter portion 90 of the guide tube 4 with the guide hub 50 comprises compression of the outer layer 24. Compression of the outer layer 24 at the distal face of the increased diameter portion 90 forms a hermetic fluid seal, preventing cerebrospinal fluid from leaking outside the cranium.

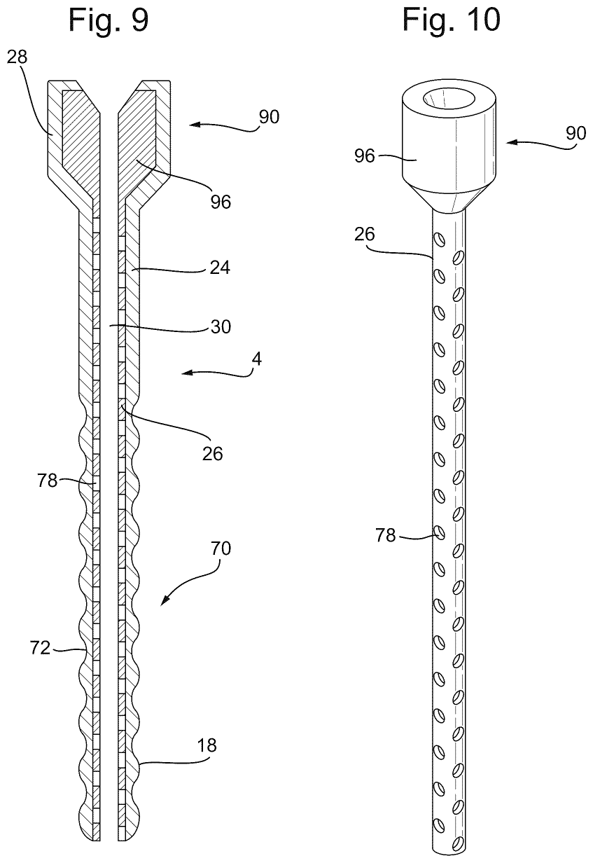

As shown in FIG. 7 and FIG. 9, the through-bore 30 of the guide tube 4 within the increased-diameter portion 90 may increase in diameter towards the proximal end of the increased-diameter portion 90. This can help to guide the fluid transfer tube 6 into the through-bore 30 and make assembly easier during use.

The increased-diameter portion 90, that may also be referred to as a flared portion, may comprise a non-compliant core 96 located inward of the outer layer 24. The non-compliant core 96 forms the increased-diameter portion 90 of the guide tube 4, because the other layers (such as the outer layer 26) will be deposited over the non-compliant core at the proximal end 18. The non-compliant core 96 may be made from PEEK. The non-compliant core 96 may be formed integrally with the inner layer 26. Alternatively, the non-compliant core 96 may be of a different material from the rest of the guide tube 4. For example, the non-compliant core 96 may be formed by an over-moulding process applying a different polymer onto the end of the guide tube 4.

The non-compliant core 96 may be over-moulded on the proximal end of the inner layer 26 and located outward of the inner layer 26. In such a case, the inner layer 24 may not extend to an extreme proximal end of the increased-diameter portion 90. The inner layer 24 may extend through at most 80%, optionally at most 60% of the increased-diameter portion 90. Thereby, the non-compliant core 96 may define the through-bore 30 at the proximal end of the guide tube 4.

The outer layer 24 may be electrospun over the external surface of the increased diameter portion 90. This increased diameter portion 90 can be useful for securing the guide tube 4 in the skull and/or forming a seal around the proximal end 28 of the guide tube 4, as discussed above.

The non-compliant core 96 may be configured to allow the passage of gas through the non-compliant core 96. For example, the non-compliant core 96 may be moulded with air venting holes or passages through it, or may be formed from a gas-porous material such as a sintered polymer. This allows the non-compliant core to contribute to the gas venting function of the guide tube 4 (discussed further below). In this case, the increase in diameter of the through-bore 30 towards the extreme proximal end of the guide tube 4 also provides increased exposed surface area of the non-compliant core 96 that can further increase the contribution of the non-compliant core 96 to the gas venting function.

The use of a porous non-compliant core 96 is also advantageous in simplifying manufacture. If the non-compliant core 96 is not porous, gas is only vented through the porous layer, which must therefore be exposed to atmosphere on the proximal face of the expanded portion 90 of the guide tube 4 to be effective. To ensure this surface is exposed requires additional manufacturing steps such as masking the proximal face of the guide tube 4 when forming other layers. Using a porous non-compliant core 96 means that the porous layer can be formed over the non-compliant core 96 without having to shield the proximal face of the expanded portion 90 of the guide tube 4, because gas can still be vented via the non-compliant core 96 and the proximal end of the through-bore 30. The resulting reduced number of steps in manufacturing the guide tube 4 greatly speeds up the manufacturing process, reduces handling, and reduces cost.

Convection enhanced delivery techniques can be used for both acute (short time) and chronic (longer term or repeated) delivery of treatment to brain tissue. For example, gene therapy may be carried out in a single treatment session, whereas treatments for other reasons such as chemotherapy may require repeated infusions into the brain (a chronic treatment regime).

In either case, but especially where a long duration or chronic treatment regime is employed, it is highly desirable that the distal end 18 of the guide tube 4 remains in place in brain tissue. The distal end 18 of the guide tube 4 is typically positioned at the proximal end of a cannula trajectory. The cannula trajectory makes up the final distal section of the trajectory to the target within the brain and traverses the target region around the target. The distal end 18 of the guide tube 4 serves to retain infusate in the target region by resisting reflux at the step 20 created between the respective diameters of the guide tube 4 and fluid transfer tube 6 as discussed above.

Fixing the proximal end 28 of the guide tube 4 to the skull (or to a guide hub 50 that is fixed to the skull) provides a relatively secure arrangement for long duration or chronic treatments. However, the brain is moveable with respect to the skull. Thus, in use the guide tube 4 may be subject to axial or even transverse forces when the brain moves. Such forces can result in the distal end 18 of the guide tube 4 repeatedly traumatising brain tissue. This may result in tissue vacuolation and the creation of a low resistance pathway at the step 20 which will augment rather than resist reflux, causing a loss of therapy from the target. A flexible guide tube 4 can reduce this problem by allowing the distal end 18 of the guide tube 4 to move relative to the proximal end 28 at which it is secured to the skull.

At least a proximal portion 38 of the guide tube 4 may be resiliently deformable along an axial direction of the guide tube 4. The resilient deformation may comprise extension and/or compression in the axial direction. At least the proximal portion 38 may comprise a material having a Poisson's ratio which is close to zero or negative when deformed (e.g., stretched or compressed) in an axial direction, i.e. such that the wall thickness does not change substantially during deformation (under small strains). For example, expanded polytetrafluoroethylene (ePTFE) or polyurethane foams can exhibit this property. More generally, many polymeric foams have close to zero Poisson's ratio, as air will tend to escape as the foam is compressed.

The proximal portion 38 may be located, in use, outside the brain (although may still be within the skull and/or cranial cavity). The proximal portion 38 may extend from outside the brain to or towards the extreme proximal end of the guide tube 4. A guide tube 4 that is resiliently deformable in at least a proximal portion 38 can lengthen and shorten in response to changing distance between the brain and a fixed proximal end 28 of the guide tube 4 at the skull. The resiliently deformable proximal portion 38 may be provided in any suitable manner. For example, the entire guide tube 4 may be resiliently deformable.

Optionally, as shown in FIGS. 2, 7, and 9, the guide tube 4 may be of laminate construction, for example comprising at least an inner layer 26 and an outer layer 24, wherein the inner layer 26 has greater stiffness than the outer layer 24. The outer layer 24 may be resiliently deformable, and in the proximal portion 38 of the guide tube 4, the inner layer 26 may be configured to provide a spring 76. FIG. 7 shows a cross-section of the guide tube 4 with inner layer 26 and outer layer 24. FIG. 8 shows the inner layer 26 separately from the outer layer 24. The proximal portion of the stiff inner layer 26 just distal to the proximal end 28 of the guide tube 4 may be in the form of a spring 76, for example formed by creating a spiral cut through the wall of the inner layer 26 along its long axis. The spring 76 may have a length of between 5 mm and 30 mm, preferably between 10 mm and 20 mm. With the proximal end 28 of the guide tube 4 fixed relative to the skull, the spring 76 of the inner layer 26 of the guide tube 4 can accommodate movement of the brain relative to the skull fixation, whilst the distal end 18 of the guide tube 4 remains fixed in its location in the brain tissue.

The outer layer 24 of the guide tube 4, being made of a resiliently deformable material, can accommodate the desired range of movement over the proximal portion 38. For example, the outer layer 24 may be of electro-spun, low durometer, polyurethane or PTFE. When formed by electrospinning, the outer layer 24 may have a high proportion of transversely aligned fibres to augment axial compliance. The outer layer 24 may be made less adherent or non-adherent to the sprung portion of the inner layer 26 of the guide tube 4 to enhance compliance of the guide tube 4 in the proximal portion 38. For example, electro-spun polyurethane has poor adherence to a smooth PEEK surface, which can be overcome by dipping the PEEK in a polyurethane solution, laser etching the surface, surface abrasion, or plasma treatment. If the inner layer 26 is formed from PEEK and is dipped in a polyurethane solution, laser etched, abraded, or plasma treated except in the sprung section, then there will be little or no adherence of the outer layer 24 to the spring 76 of the inner layer 26.

When the guide tube 4 with a sprung proximal portion 38 is delivered into the brain parenchyma using a delivery probe (as described below), the coaxial force applied to the guide tube 4 will compress the spring 76 so that the guide tube's column strength is sufficient to deliver it to the desired target. In situ, with the delivery probe removed and replaced by the fluid transfer tube 6, the distal end 18 of the guide tube 4 will remain fixed at its location in the brain, even though the fluid transfer tube 6 may move relative to the guide tube 4. Relative movement of the fluid transfer tube 6 and guide tube 4 may augment reflux along the fluid transfer tube 6. However, this will be confined to the target volume because the step 20 created by the distal end of the guide tube 4 and the sealing region 70 of the guide tube 4, that is the primary control of reflux, will be at a fixed position at the brain target. The sealing region 70 will also assist in retaining the distal end 18 of the guide tube 4 at its target position, because the reduced-pressure region 72 will act to key into the brain tissue.

Instead of providing a sprung section of the inner layer 26 as in FIGS. 7 and 8, the inner layer 26 may be reduced or even absent in the proximal portion 38. The guide tube 4 may comprise at least an inner layer 26 and an outer layer 24, wherein the inner layer 26 has greater stiffness than the outer layer 26 and the outer layer 26 is resiliently deformable as before.

The inner layer 26 may extend proximally from the distal end 18 of the guide tube 4, but not extend to the extreme proximal end of the guide tube. Rather, the inner layer 26 does not extend into the proximal portion 38 of the guide tube 4. Thereby, the proximal portion 38 of the guide tube, without the inner layer 26, extends from the extreme proximal end of the guide tube 4 to the beginning of the inner layer 26. The proximal portion 38 can be sized to extend in use from the skull of a patient to or towards the outer layer of the brain. Since the relatively stiff inner layer 26 is absent from the proximal portion 38, the proximal portion 38 can lengthen and shorten allowing the distal end of the guide tube to stay in place in the brain tissue.

When the guide tube 4 is flexible, the fluid transfer tube 6 may also be flexible. For example, the fluid transfer tube 6 may comprise or be made from a flexible material. This allows the fluid transfer tube 6 to move and retain its intended position at the target when the brain moves relative to the skull, particularly during lateral movement of the brain.

The guide tube 4 may be constructed of a non-homogeneous material and/or of a plurality of materials with different properties. This applies mainly to guide tubes 4 secured to the skull such as shown in FIGS. 7-15. While guide tubes 4 such as shown in FIG. 5 may have multiple layers, they are more often formed from a single homogeneous layer.

For example, as mentioned above, the guide tube 4 may be of laminate construction. This can provide a desired combination of features as will be discussed further below, including an over-mould to create a flared or enlarged proximal end, the property of air venting along the length of the guide tube 4, and a fine and grooved guide tube 4 with sufficient stiffness in the axial direction to maintain structural integrity when delivered into tissue.

In some situations, for example when using a rigid guide tube 4 such as shown in FIG. 5, the fluid transfer tube 6 and guide tube 4 may be assembled together and delivered into the brain together. In other situations, for example when using guide tubes 4 such as those in FIGS. 7-15 that are secured to the skull, the distal end 10 of the fluid transfer tube 4 may be passed into the through-bore 30 of the guide tube 4 after insertion of the guide tube 4 into the brain, passing through the proximal end 28 of the guide tube 4 to the distal end 18 and being delivered to the target in the brain parenchyma. During the passage of the fluid transfer tube 6 through the through-bore 30 of the guide tube 4, a column of air in the guide tube 4 is forced distally with a piston-like action. This can lead to tearing of brain tissue in the target region.

To mitigate this, the guide tube 4 may be configured to vent gas from the through-bore 30 to a proximal end 28 of the guide tube 4, preferably through an inner surface of the through-bore 30.

The guide tube 4 may comprise a porous layer to allow passage of air, the porous layer comprising a hydrophobic material. The porous layer may be an outermost layer of the guide tube 4. To maintain the air porosity of the guide tube 4, the porous layer is in gaseous communication with the through-bore 30. Due to the porous layer, air is driven into the walls of the guide tube 4 and passes out to the atmosphere through its proximal end 28 in preference to being driven into the brain parenchyma at the target region. This avoids tearing brain tissue. Venting of air from the guide tube 4 to atmosphere may be stopped when the fluid transfer tube 6 is fully inserted into the guide tube 4. At this point, a distal face of a depth-controlling stop 40 on the fluid transfer tube 6, or an interposing washer, may engage with the proximal face of the proximal portion of the guide tube 4 to create a seal.

The porous layer may comprise a hydrophobic material or a superhydrophobic material. By hydrophobic is meant that the static contact angle θ at the liquid-vapour interface of a water droplet on the surface of the material is greater than 90°. A superhydrophobic material has a static contact angle θ >150°. Alternatively to using a porous layer composed entirely of hydrophobic material, the porous layer may comprise an inner air-permeable layer, which may not be hydrophobic or superhydrophobic. The inner air-permeable layer is then sealed from liquid ingress through an outside diameter of the guide tube 4 by a liquid-impermeable layer. The liquid-impermeable layer may be made of hydrophobic or superhydrophobic material.

An additional advantage of the use of a porous layer for at least the outermost layer of the guide tube 4 is that it may provide integration with brain tissue over time, because the brain tissue may enter the porous structure. This can aid in securing the guide tube 4 in its desired location, even if the compressive force between brain tissue and guide tube 4 reduces over time. Integration of the guide tube 4 and brain tissue can remove the sharply defined guide tube-tissue interface, and thereby will further reduce the likelihood of reflux.

Alternatively, the guide tube 4 may comprise a fluid-impermeable covering layer, which may provide advantages such as reduced trauma upon its insertion and improved sealing. Preferably the covering layer is lubricious to reduce trauma on insertion as discussed above. The covering layer may be provided over the porous layer. The covering layer is preferably not provided over at least part of a proximal surface of the guide tube 4 so that it does not prevent air venting to atmosphere from the porous layer at the proximal end of the guide tube 4 during use.

Suitable materials of construction for at least the porous layer of the guide tube 4 include at least one of sintered polytetrafluoroethylene (PTFE), sintered polyurethane, expanded PTFE, silicone foam, polyurethane foam, shape memory polymer, microporous hollow extruded-polymer fibres, or electrospun polymer. The microporous hollow extruded-polymer fibres and/or the electrospun polymer may comprise at least one of polytetrafluoroethylene, polyvinylidene difluoride, polyurethane, polypropylene, and copolymers thereof. Shape memory polymers may be employed. Electrospun polyurethane is air porous and can be made in hydrophobic, superhydrophobic, or non-hydrophobic forms. Where foams are employed, at least a portion of the cells of the foam are open cell to allow passage of air through the bulk material. All or substantially all of a foam construction outer layer 24 or porous layer of the guide tube 4 may be an open cell foam.

As mentioned above, it may be necessary to cut the guide tube 4 to a desired length prior to implantation of the guide tube 4 into the brain. The resiliently deformable outer layer 24 may make cutting of the guide tube 4 more difficult, because the outer layer 24 of the guide tube 4 will tend to deform when a cutting force is applied. To address this, the guide tube 4 may be provided as part of a package. The package may comprise the guide tube 4 and a packaging tube, wherein the guide tube 4 is provided within the packaging tube. The packaging tube may have a stiffness that is greater than that of the outer layer 24 of the guide tube 4. The packaging tube holds the guide tube 4 in place and reduces its tendency to deform when a cutting force is applied, ensuring a clean axial cut of the distal end 18 of the guide tube 4. Once the guide tube 4 has been cut to length, it can be removed from the package immediately prior to its insertion into the brain. The packaging tube also reduces direct handling of the outer surface of the guide tube 4 before implantation, thereby reducing the chance of contamination of the guide tube 4, for example with pathogens. The package may further comprise a stylet within the through-bore 30 of the guide tube 4 as mentioned above. The stylet further reduces deformation of the guide tube 4 during cutting.

Where the guide tube 4 is constructed of a non-homogeneous material and/or a plurality of materials with different properties, the stiffness in particular may vary, such that the guide tube, and in particular the porous layer, comprises a non-homogeneous material having varying stiffness and/or a plurality of materials having different stiffnesses. For example, the stiffness of the porous layer may be greater at or near the through-bore 30. The porous layer may be constructed of electrospun polymer or a foam having increasing density from the outside radially inwards towards the through-bore 30, or a region of increased density at or near the through-bore 30. The increased density provides increased stiffness, thereby providing support to a more porous and compliant outer region.

The stiffening may be provided along the entire or substantially the entire length of the guide tube, as shown in the examples of FIGS. 9 and 10. However, in some embodiments of the invention, the provision of stiffening may be varied along the length of the guide tube 4. For example, as discussed above, there may be reduced stiffening or even no additional stiffening provided at the proximal end 28 of the guide tube 4 to permit axial deformation of the guide tube 4.

Alternatively or additionally, and as shown in FIGS. 7-10, the guide tube 4 may have a laminated structure with a stiffer inner layer 26 (or layers) at or near the through-bore 30. In this case, the porous layer may comprise at least an inner layer 26 and an outer layer 24, wherein the inner layer 26 has greater stiffness than the outer layer 24. For example, the stiffer inner layer 26 may provide the surface of the through-bore 30. The inner layer 26 may be an innermost layer of the guide tube 4, and thereby provide the surface of the through-bore 30. The stiffer inner layer 26 provides support to a more compliant outer layer 24. The porous outer layer 24 may be electro-spun onto the relatively stiff inner layer 26 formed for example of electrospun or microperforated PEEK tubing.

The stiffer inner layer 26 may be of a hydrophobic or superhydrophobic material to aid in avoiding liquid seepage through the guide tube structure. However, this is not essential and one or more layers may not be hydrophobic or superhydrophobic.

The inner layer 26 may comprise a polymer material, for example at least one of: polyether ether ketone; nylon; polyurethane; polyester; a fluoropolymer such as polytetrafluoroethylene; a polymeric perfluoroether such as perfluoroalkoxy alkane, polyvinylidene difluoride, or fluorinated ethylene propylene; liquid crystal polymers; and mixtures or copolymers thereof. The inner layer 26 may be manufactured by a process comprising at least one of: sintering, extrusion with particulate leaching, micro-perforating a tube by drilling or by laser; weaving, braiding or electrospinning polymer fibres about a cylindrical former to form a tube of porous polymer sheet material; and 3D printing a polymer in a porous form.

To maintain the air porosity of the guide tube 4, it is preferable that the porous outer layer 24 is in gaseous communication with the through-bore 30 along a substantial part, for example at least 25%, optionally at least 50%, of the length of the guide tube 4. Where the guide tube has a multi-layer or laminated structure, the stiffer inner layer 26 (or layers) at or near the through-bore 30 may be configured to allow passage of air through the inner layer 26. This can allow air in the through-bore 30 to vent through the porous material of the outer layer 24 of the guide tube 4. However, if the inner layer 26 is not configured to allow passage of air, air driven down the through-bore 30 may still be removed from the distal end 18 of the guide tube 4 when it contacts the extreme distal end of the porous outer layer 24, and travel back up through the porous outer layer 24 to the proximal end 28 of the guide tube 4.

The inner layer 26 may comprise a material porous to air. Alternatively, the inner layer may be of a naturally non-porous material but can be manufactured to allow passage of air in various ways. For example, as shown in FIGS. 6-10, the inner layer 26 may comprise a plurality of holes 78 through the inner layer 26. The holes 78 may be provided over at least 25%, optionally at least 50%, optionally at least 75% of the length of the inner layer 26. The holes 78 may be formed by micro-perforating the material used to form the inner layer 26, by drilling, or using a laser. The porous inner layer 26 may also be formed by weaving, braiding or electrospinning polymer fibres about a cylindrical former to form a tube of porous polymer sheet material. 3D printing could also be used to produce a porous inner layer 26.

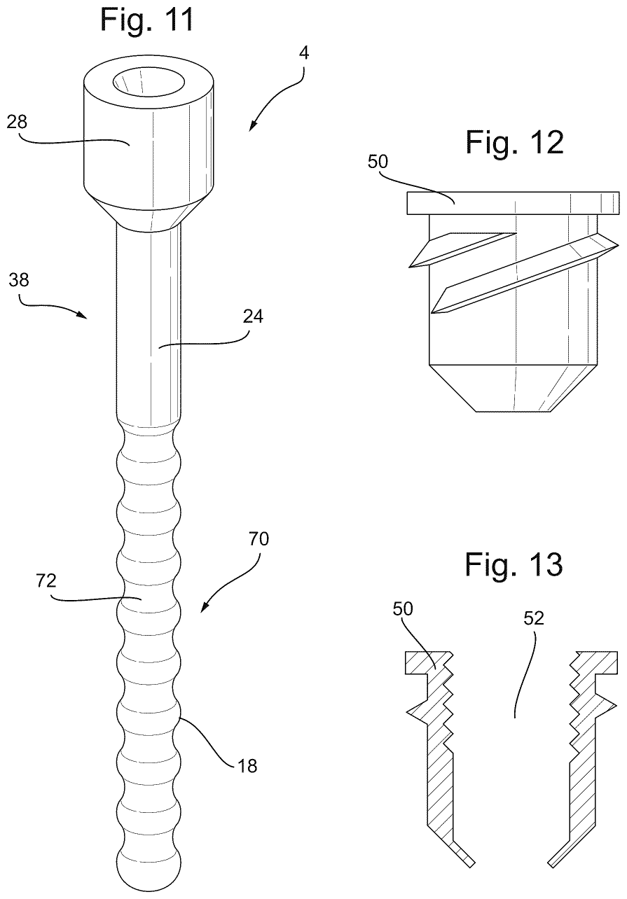

The inner layer 26 may comprise a proximal, over-moulded increased diameter portion 90 such as discussed above, for example made from PEEK, that may also be referred to as a flared portion. The increased diameter portion 90 may be of a different material from the rest of the guide tube 4. For example, the increased diameter portion 90 may be formed by an overmolding process applying a different polymer onto the end of the guide tube 4. The air porous outer layer 24 may be electrospun over the external surface of the increased diameter portion 90, as shown for example in FIG. 11. This increased diameter portion 90 can be useful for securing the guide tube 4 in the skull and/or forming a seal around the proximal end 28 of the guide tube 4, as will be discussed further below.

The use of electrospinning to construct the guide tube 4 confers the advantage of being able to tightly control the device's form, as well as its mechanical and chemical properties. For example, a fine tube which is both stiff and air porous may be electrospun from a polymer such as PEEK or LCP by collecting fibres on a rotating metal rod. By modulating the rate of rotation of the rod collector and the use of shaped, guiding, and shielding electrodes, layering of fibres along both the longitudinal and transverse axis can be achieved to control porosity and maximise its column strength.

The outer layer 24 of the guide tube 4 may be electrospun over the stiffer inner layer 26, for example with polymers having preferred properties such as hydrophobicity, super-hydrophobicity, tissue adherence, greater compliance, etc.

The reduced pressure regions 62 may be formed in the process of sintering, moulding or electrospinning the porous layer 24. For example, the reduced-pressure regions 72 can be formed by creating a plurality of circumferential rings with circumferentially aligned fibres along the long axis of the guide tube 4. This can be achieved, for example, by electrospinning over a rotating metal rod with circumferential grooves or insulated rings along its length to create sections of high current density where fibres will be maximally deposited, and/or by deploying focusing, steering, and guiding electrodes in combination with tightly controlled rod rotation rates.

The reduced-pressure regions 72 could also be formed by creating a guide tube 4 having a uniform diameter, and then processing the guide tube 4 to form the sealing region 70 using one or more heated rollers. The heated roller(s) may have circumferential protuberances with a width and spacing to match the desired configuration of the reduced-pressure regions 72 and would have their axis of rotation parallel to the guide tube axis. The rollers can be simultaneously pressed into the outside of the guide tube 4 to reduce its diameter in the reduced-pressure regions 72. The heated rollers and/or the guide tube 4 may be rotated during this process. Where the guide tube 4 (or at least its outer layer 24) is formed by electrospinning, the heated rollers may act to compress and fuse together the fibres of the outer layer 24, thereby reducing the diameter of the guide tube 4 in that area.

It is possible to use three parallel shafts of heated rollers, place the guide tube between them and apply the rollers from three sides of the parallel guide tube. The rollers can then be rotated in the same sense (i.e. clockwise or counter-clockwise), and this will serve to rotate the guide tube about its longitudinal axis and create a groove or series of grooves around the whole circumference of the guide tube.

The reduced pressure regions 72 may be provided by a covering layer such as discussed above. For example, the covering layer may comprise a non-porous and lubricious heat shrink tube that is applied over a resiliently deformable porous layer and focally heat shrunk along its axis when it is rolled between heated rollers with transverse parallel ridges on their surface. For example, the reduced pressure regions 72 could be formed by sliding a heat shrink polymer tube over the external surface of an electrospun guide tube, and then placing the construct between parallel heated rollers as described above. The rollers would apply a variable heat and compression on the external surface of the heat shrink, creating a series of grooves on the outer surface of the guide tube. The heat shrink material could be Fluorinated Ethylene Propylene (FEP), Polytetrafluoroethylene (PTFE), Polyethylene Terephthalate (PET) or Polyolefin. Alternatively, a heat shrink polymer tube with spaced annular thickenings along its wall could be directly applied to the external surface of an electrospun guide tube and uniformly heated to shrink and mechanically lock onto the guide tube. The profiled external surface of the heat shrink may not only provide the reduced pressure regions but may in addition provide a lubricious and fluid impermeable external surface to the guide tube.

A rigid and air porous guide tube could be formed by sintering a hydrophobic thermoplastic polymer, for example, Polytetrafluoroethylene (PTFE), Polyethylene (PE), Polypropylene (PP), High Density Polyethylene (HDPE), Ultra-High-Molecular-Weight Polyethylene, (UHMWPE) and Polyvinyl Chloride (PVDF). The sintering mould could contain a core pin held under tension to maintain its straightness, or manufactured from a highly rigid material, e.g., Tungsten Carbide. The sintering mould could contain circumferential grooves on its inner surface, which will form a guide tube with reduced-pressure regions on its outer surface. A thermoplastic polymer tube may be heat-shrunk over the external surface of the profiled sintered guide tube to provide a lubricious and fluid impermeable surface. Alternatively, a thermoplastic polymer tube with spaced annular thickenings along its wall could be heat shrunk onto the external surface of a guide tube made of a sintered polymer which has a uniform diameter to create a profiled guide tube with reduced pressure regions.

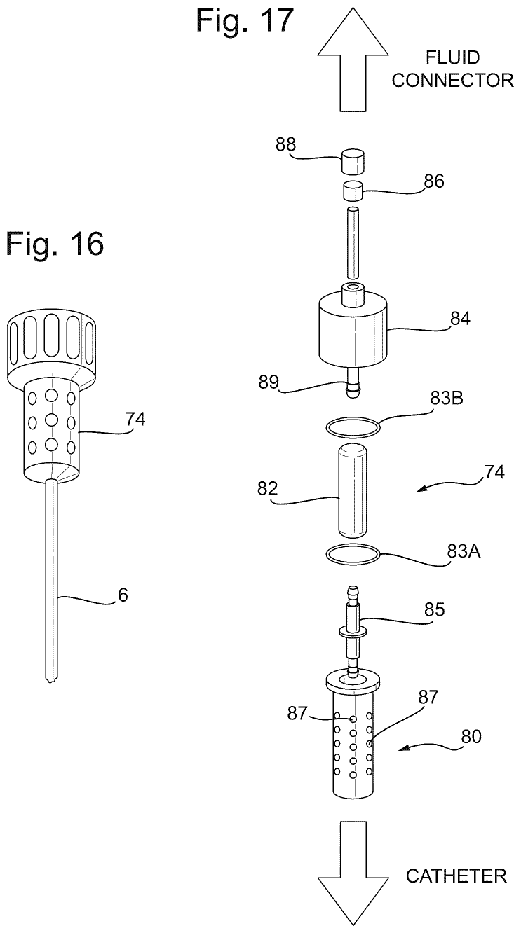

It is important that air not be delivered into the brain through the fluid transfer tube 6, and to reduce this risk, the fluid transfer tube 6 may comprise a bubble vent 74. The bubble vent 74 is preferably provided at a proximal end of the fluid transfer tube 6, as shown in FIG. 16. The configuration and component parts of the bubble vent 74 are described below and illustrated in FIG. 16. The bubble vent 74 is configured to prevent gas from entering the fluid transfer tube 6. The bubble vent 74 mitigates the risk of bubbles entering the brain if they have come out of solution in the infusate or have been entrained in the infusate during the connection and/or disconnection of a delivery system that is used to deliver the infusate to the fluid transfer tube 6, such as a dispenser, infusion line, and/or pump. Bubbles infused into brain tissue can tear the tissue and disrupt distribution of the therapeutic fluid/infusate. The bubble vent 74 may be permanently joined to and/or integrally formed with the fluid transfer tube 6, which further reduces the chance of bubbles being entrained during connection of the fluid transfer tube to the delivery system. The bubble vent 74 is preferably provided integrally with the cannula 6, as shown in FIG. 16. The bubble vent 74 may additionally or alternatively be configured to prevent pathogens (for example micro-organisms such as bacteria) from entering the fluid transfer tube 6. This reduces the risk of intracranial infections occurring due to the treatment.

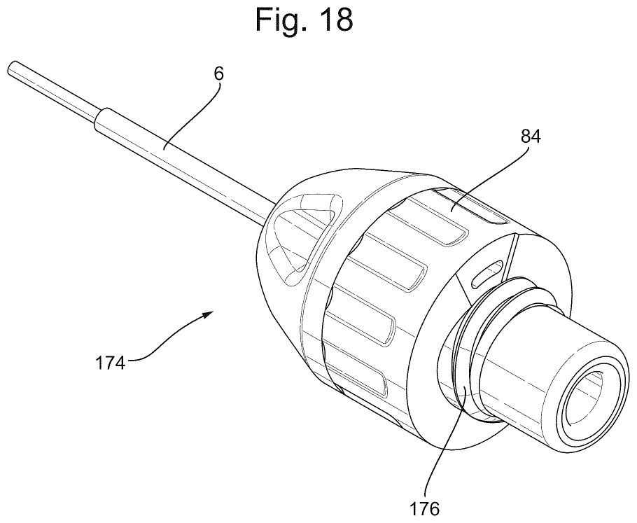

FIG. 17 illustrates an exploded view of the bubble vent 74, configured for attachment to the cannula 6 and a fluid connector of the delivery system. The bubble vent 74 includes a perforated filter guard 80, a bubble filter 82, retaining rings 83A, 83B, a retaining cap 84, a septum stopper 86 and a septum cap 88. The bubble vent 74 may comprise a low volume bubble filter 82, for example made from expanded polytetrafluoroethylene (ePTFE). The bubble filter 82 may have a hydrophobic (optionally superhydrophobic), gas permeable, microporous structure configured to remove bubbles from the flowing therapeutic fluid/infusate. The bubble filter 82 may be effective to remove bubbles at flow rates of less than or equal to 30 μl/min. The bubble vent 74 may further comprise a filter guard 80. In the illustrated example the bubble filter 82 is effective to remove bubbles at flow rates of less than or equal to 30 μl/min. The filter 82 is contained in the perforated filter guard 80 and in the illustrated example the bubble filter 82 is received over a hollow post 85 and is retained by a retainer ring 83A to the hollow post 85, which is positioned concentric to the filter guard 80.

The filter guard 80 may comprise a hollow shell that includes a distribution of a plurality of perforations 87 (small holes) around the shell wall that facilitate degassing the fluid/infusate as it flows through the bubble vent 74 to the fluid transfer tube 6. The filter guard 80 may also guard/protect the bubble filter 82 against damage. The combination of the bubble filter 82 and the filter guard 80 facilitates dispersion of entrained air/bubbles from the fluid flow before the fluid passes into the fluid transfer tube 6.

The bubble 74 vent may comprise a retaining cap 84, which connects with the filter guard 80 to complete the assembly of the bubble vent 74 and to contain the bubble filter 82 within the bubble vent 74. The retaining cap 84 may include a hollow retaining post 89 and a retainer ring 83B which engage with the bubble filter 82 to ensure the bubble filter 82 is correctly positioned and retained in the filter guard 80 to ensure efficient functionality of the bubble filter 82 during use. The retaining cap 84 may include a septum stopper 86, which provides a sealed unit until the septum 86 is pierced by a hollow needle to provide fluid connection to the fluid transfer tube 6. The septum stopper 86 may be retained under compression by a septum cap 88. The retaining cap 84 and filter guard 80 may be joined by a snap fit connection. However, alternative arrangements could be used to join them together, for example a threaded connection, welded connection, glued connection etc.

The bubble vent 74 incorporating the bubble filter 82 reduces the risk of air being delivered e.g. to the brain with the fluid containing therapeutic agent/infusate. It will be appreciated fluid containing air/bubbles will be space occupying and therefore is capable of stretching and tearing brain tissue whilst also disrupting delivery/distribution of the therapeutic agent/infusate. The bubble vent 74 can also act to filter out pathogens, including bacteria and other microorganisms from the fluid.

FIGS. 18 to 21 show an alternative design of bubble vent 174. FIG. 18 shows the bubble vent 174 in its assembled state ready for use. Similar to the bubble vent 74, the bubble vent 174 comprises a retaining cap 84. The retaining cap 84 comprises a proximal connector 176, for example a threaded connector, for connection to a fluid connector of the delivery system.

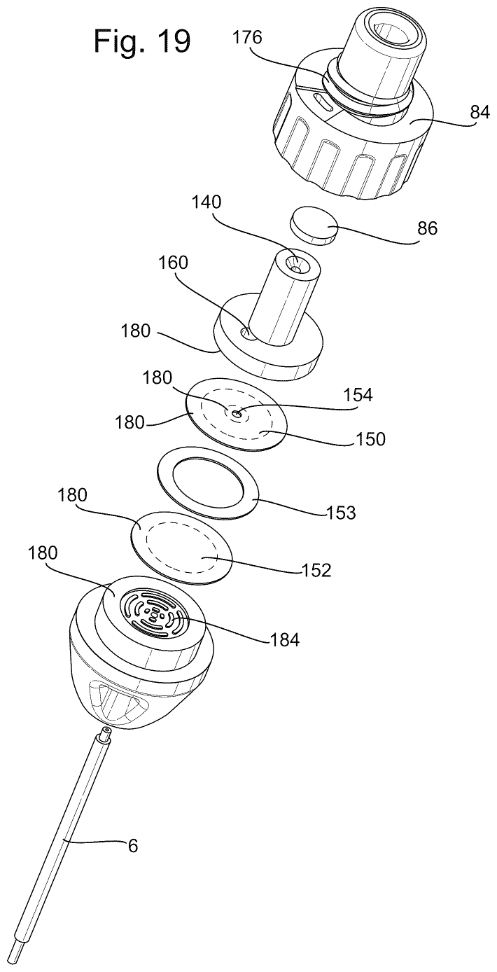

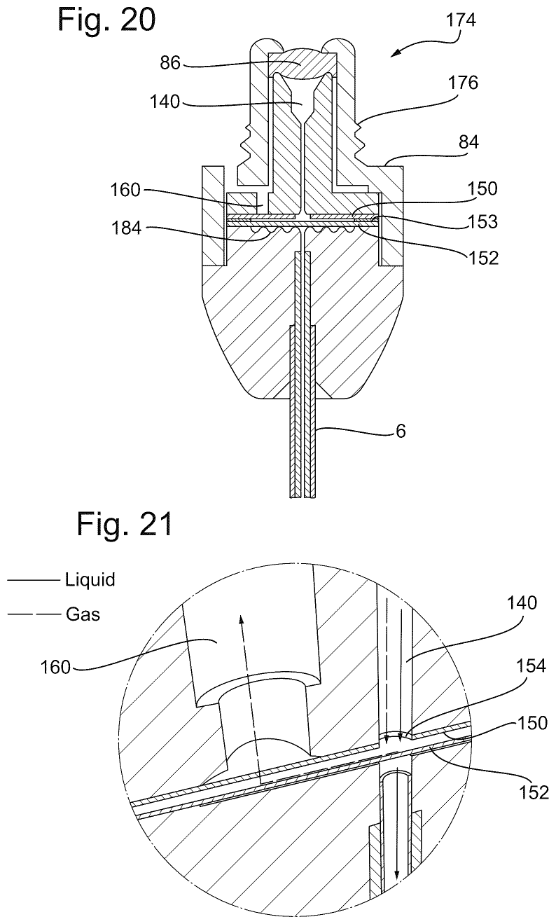

FIG. 19 shows an exploded view of the bubble vent 174, and FIG. 20 shows a cross-sectional view. The proximal connector 176 may comprise a septum 86 to seal the proximal connector 176 until the septum 86 is pierced, for example by a hollow needle. The bubble vent 174 comprises a fluid passage 140 fluidly connecting the proximal connector 176 and the fluid transfer tube 6. In this design, the bubble vent comprises a first membrane 150 and a second membrane 152. The first membrane 150 and the second membrane 152 are positioned between a distal end of the fluid passage 140 and a proximal end of the fluid transfer tube 6. The first membrane 150 and the second membrane 152 may be substantially parallel to one another, and may be substantially perpendicular to an axis of the fluid passage 140. The first membrane 150 is positioned closer to the distal end of the fluid passage 140 than the second membrane 152, such that fluid entering the bubble vent 174 through the septum 86 reaches the first membrane 150 before the second membrane 152. An annular washer 153 may be positioned between the first membrane 150 and the second membrane 152 that forms a peripheral fluid seal between the membranes and the housing of the connector 174 and separates the membranes centrally to create a cylindrical gap between them. The cylindrical gap may have a diameter of between 2 mm and 6 mm but is most preferably 4 mm. The gap may separate the membranes 150 and 152 by 0.05 mm to 0.2 mm but most preferably by 0.1 mm. The first membrane 150 and the second membrane 152 may be connected to one another and to the other components of the bubble vent 174 via connection surfaces 180. The connection surfaces 180 may be joined by any suitable method, for example using ultrasonic welding or using an adhesive layer. The bubble vent 174 may comprise a support member 184 to support the distal surface of the second membrane 152 and allow liquid that has passed through the second membrane 152 to more easily reach the cannula 70.

The first membrane 150 is hydrophobic and gas permeable. A hole 154 is provided in the first membrane 150 where the fluid passage 140 meets the first membrane 150, such that fluid from the fluid passage 140 can pass through the first membrane 150 via the hole 154. The septum sealed connector 174 may comprise a support member to support the proximal surface of the first membrane 150 and annular connection surfaces to attach the membrane around its periphery and around its central hole 154 (not shown in FIG. 19). The second membrane 152 is liquid permeable and preferably hydrophilic. It is not essential that the second membrane 152 is hydrophilic, however gas venting works most efficiently using the combination of a hydrophobic and a hydrophilic membrane. Use of the hydrophobic first membrane 150 alone could result in air being drawn from the atmosphere through the first membrane 150 and into the infusate if the pressure in the line falls below atmospheric pressure. This can happen if the connector is elevated above the head by more than 10-25 cm (depending on intracranial pressure). The second membrane 152 being hydrophilic prevents air ingress into the brain even in such situations. The second membrane 152 is impermeable to gas and bacteria. No hole is provided in the second membrane 152, such that fluid from the fluid passage 140 must pass through the material of the second membrane 152 to reach the fluid transfer tube 6. One or more vent holes 160 (for example, two vent holes in the example of FIG. 9) are provided in the bubble vent 174 on a proximal side of the first membrane 150. No holes are provided in the first membrane 150 where the vent holes 160 meet the first membrane 150, such that fluid from the fluid passage 140 must pass through the material of the first membrane 150 to reach the vent holes 160.

The operation of the bubble vent 174 is demonstrated in the close-up view of FIG. 21. A mixture of liquid and gas (for example an infusate to be delivered to a patient's brain via the fluid transfer tube 6 with some entrained bubbles of air) enters the bubble vent 174 via the septum 86 and the fluid passage 140. The mixture passes through the first membrane 150 via the hole 154. The liquid is drawn to the hydrophilic second membrane 152, and soaks through the second membrane 152 (which is liquid permeable) into the cannula. The layer of liquid and the second membrane 152 form a barrier preventing gas from passing into the fluid transfer tube 6. The gas will pass along the air gap between the first membrane 150 and the second membrane 152, and can escape through the gas-permeable first membrane 150 at the position of one of the vents 160. The hydrophobic nature of the first membrane 150 repels liquid and prevents the liquid forming a similar barrier as on the second membrane 152, thereby allowing the gas to pass out of the bubble vent 174 via the vent holes 160.

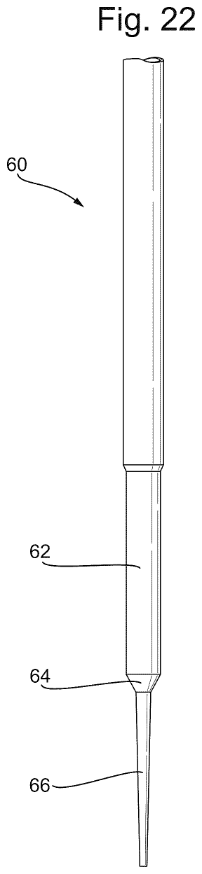

The neurosurgical apparatus comprising the guide tube 4 may further comprise a probe 60 configured for insertion into brain tissue, such as that shown in FIG. 22. The probe may be a track forming probe. The probe 60 is passed onto the brain tissue prior to insertion of the guide tube 6 to form a track for insertion of the guide tube 4. This may not be necessary where a rigid guide tube 4 is used, but may still be preferable to form the track in a controlled and minimally traumatic manner.

The probe 60 comprises a rod 62 at a distal end of the probe 60, the rod 62 having a distal end 64 that is rounded or conical in shape. The rod 62 is further provided with a spike 66 extending axially from the distal end of the rod 62. The spike 66 has a narrower diameter than the rod 62 and has an extreme distal end configured for dissecting brain tissue. The spike 66 may be provided with a rounded extreme distal end. The spike 66 may taper from the point at which it joins the rod 62 at the rounded distal end of the rod to the extreme distal end of the spike 66. The spike 66 may have a maximum diameter that it at most, preferably less than, the diameter of the fluid transfer tube 6.

The probe 60 should have an outside diameter which is at most the same as the outer diameter of the guide tube 4. A section 62 of the probe 60 that will form the section of the track in which the sealing region 70 will be present in use should have a diameter at most that of the guide tube 4 in the reduced-pressure regions 72. For example, to achieve this the probe 60 may have a smaller diameter at its distal end than at its proximal end, as shown in FIG. 22. The probe 60, excluding its smaller diameter distal end 64 and the spike 66, may otherwise have an outside diameter which is the same or no greater than the outside diameter of the guide tube 4.

In use, the spike 66 at the distal end of the probe 60 can dissect tissue with reduced trauma as it is inserted. This creates a reduced resistance pathway for the rounded or conical distal end 64 which dilates the tissue to the larger diameter of the rod 62, thereby creating a track with minimal tissue trauma.

For creating a track for use with the guide tubes described above, the probe 60 may have a diameter of 1.2 mm or less. The rod 62 may have a diameter smaller than the diameter of the probe 60 proximal to the rod 62. The rod 62 may have a diameter that is at least 0.1 mm less than a diameter of the probe 60 proximal to the rod 62, for example the rod 62 may have a diameter of 1.1 mm or less. The rod 62 may have a length of at most a length of the sealing region 70. The rod 62 may have a length approximately equal to a length of the sealing region 72. The spike 66 may be 4 mm to 5 mm long, and may taper from 0.6 mm or 0.5 mm where it joins the rod 62 to 0.3 mm or 0.2 mm at its extreme distal end. For example, the spike 66 may taper from 0.5 mm where it joins the rod 62 to 0.3 mm at its extreme distal end. Where the probe 60 has a 1.2 mm outer diameter, the track produced in brain tissue will tend to be of a slightly smaller diameter after removal of the probe, for example 1.1 mm.

The probe 60 may be made from a rigid material such as hardened stainless steel or tungsten carbide. The probe 60 may have a coating to increase its lubriciousness, for example a coating of PTFE or Parylene. The profile of the probe 60 is designed to pass through the brain tissues to a target with precision and minimal trauma so that the subsequent insertion of the guide tube 4 is relatively atraumatic. It is also designed to form the track by gentle dilation of the tissue to allow for creation of a tissue seal, rather than fracturing of the tissue. In addition to providing a means for creating a track for the guide tube 4, the spike 66 of the probe may be used to form a track for the fluid transfer tube 6 beyond the end of the guide tube 4.

The probe 60 may be provided as part of a kit for convection enhanced delivery of an infusate to the brain parenchyma. The kit may include the probe 60 for insertion into brain tissue and a guide tube 4. The kit may include a guide hub 50 and/or a fluid transfer tube 6 for fitting through the guide tube into the brain. The kit may further include a delivery probe discussed below.

Particularly where the guide tube 4 is flexible, the guide tube 4 may be inserted into the brain over a delivery probe using stereotactic guidance, before the proximal end 28 of the guide tube 4 fixed at or near the skull, preferably within a guide hub 50.

The delivery probe is configured for insertion into the through-bore 30 of the guide tube 4. The guide tube 4 is inserted into the brain when mounted on the delivery probe. To allow the delivery probe to drive the guide tube 4 into place it may be shaped to engage the proximal end 28 of the guide tube 4. For example, the delivery probe may have an increased diameter at a proximal end creating a ‘step’ in the outer surface of the delivery probe that engages the extreme proximal end of the guide tube 4. Where the guide tube 4 has a laminated structure and a proximal portion 38 without an inner layer 26, as discussed above, the delivery probe may have two steps. A more distal step engages the top of the inner layer 26 to apply an insertion force. The other, more proximal step engages the extreme end of the proximal portion 38, and may be positioned to compress the proximal portion. Typically, this compression will only be slight e.g., by 1-3 mm if the proximal portion 38 is about 1-1.5 cm long. This compression on insertion provides a slight preload to the proximal portion 38, so it can lengthen readily if the skull to brain distance lengthens.

Once the delivery probe delivers the guide tube 4 into the brain, the distal end 18 of the guide tube 4 will be located at its pre-determined target location in the brain. A distal end of the delivery probe may be configured for dissecting brain tissue. In this case, the delivery probe may be advanced further along the same trajectory as the guide tube 4 so that its distal end reaches the planned location for the fluid transfer tube 6, forming a track for the fluid transfer tube 6. The delivery probe is then withdrawn, leaving the guide tube 4 in situ and a distal track to accommodate the fluid transfer tube.

Methods of surgery making use of the apparatus described herein are described below. A method for implantation of a guide tube for convection-enhanced delivery of an infusate to brain parenchyma comprises inserting the guide tube 4 into the brain until the distal end of the guide tube 4 reaches a planned location in the brain. The guide tube 4 comprises a through-bore 30 for passage of a fluid transfer tube 6 and a distal end 18 comprising a sealing region 70. The guide tube 4 may be a guide tube 4 such as discussed above. The guide tube 4 may be inserted into the brain with the aid of a delivery probe passing through the through-bore 30 of the guide tube 4 as discussed above, such that a distal end of the delivery probe is at or just beyond the distal end 18 of the guide tube 4. The inserting of the guide tube 4 may be carried out with a delivery probe within the through-bore 30, such that a distal end 18 of the delivery probe extends to or just beyond the distal end 18 of the guide tube 4. The method may further comprise cutting the guide tube 4 to a desired insertion length with respect to a fixation point in the skull prior to insertion of the guide tube 4 into the guide tube track.

The method further comprises compressing brain tissue adjacent to said sealing region 70 to form a reflux-inhibiting seal around the distal end 18 of the guide tube 4, and forming a reduced-pressure region 72 in which the compression of the brain tissue is lower than the compression of brain tissue in both a proximally adjacent region and a distally adjacent region to the reduced-pressure region.

The compressing of the brain tissue adjacent to the sealing region 70 to form the reflux-inhibiting seal may also form the reduced-pressure region 72, i.e. so that the reflux-inhibiting seal is formed at least in part by the reduced-pressure region 72. Compression of the brain tissue and formation of the reduced-pressure region 72 may be achieved using one or a plurality of features on an outer diameter of the guide tube 4. The feature(s) may comprise a change in the outer diameter of the guide tube 4 in the sealing region 70, such that the outer diameter of the guide tube 4 varies in the sealing region 70. For example, the reduced-pressure region(s) 72 may be provided by one or more grooves in an outer surface of the guide tube 4, one or more protrusions 94 from an outer surface of the guide tube 4, or a combination of one or more protrusions and one or more grooves.

The method may comprise, prior to inserting the guide tube into the brain, inserting a probe 60 into the brain parenchyma to form a guide tube track. The guide tube track extends to the planned location in the brain at which a distal end of the guide tube 4 is to be placed. The guide tube 4 may have a diameter at least that of the probe 60. The probe 60 may comprise a rod 62 having a distal end 64 that is rounded or conical in shape, and a spike 66 extending axially from the distal end 64 of the rod 62, as discussed above. The spike 66 may have a narrower diameter than the rod 62 and having an extreme distal end configured for dissecting brain tissue.

Once the guide tube 4 has been implanted, it can be used to deliver an infusate to the brain. A method of convection-enhanced delivery of an infusate to brain parenchyma may comprise implanting the guide tube 4 into the brain as described above.

The method may further comprise advancing a delivery probe through the through-bore 30 along the axis of the guide tube 4 to form a fluid transfer tube track in the brain tissue located at the distal end 18 of the guide tube 4. This fluid transfer tube track extending through the brain tissue distal of the guide tube 4 will accommodate the fluid transfer tube 6. The fluid transfer tube track may have a narrower diameter than the guide tube track. Once the distal end of the guide tube 4 reaches the planned location and the fluid transfer tube track has been formed, the delivery probe can be removed leaving the guide tube 4 in place within the brain.

As an alternative to advancing a delivery probe through the through-bore 30, the fluid transfer tube track may be created by i) removing the delivery probe when the distal end 18 of the guide tube 4 is at its planned position; ii) inserting a track making probe down the guide tube 4 along the trajectory and beyond the distal end 18 of the guide tube 4 to create the fluid transfer tube track through the brain tissue; and iii) removing the track making probe.

The method may further comprise passing a fluid transfer tube 6 through the through-bore 30 of the guide tube 4 and into the brain along the fluid transfer tube track, and delivering the infusate into the brain via the fluid transfer tube. The infusate may carry any suitable therapeutic agent, inert fluid, imaging agent or diagnostic agent that can be delivered into the brain tissue via a suitable biologically inert fluid. In this case, the guide tube 4 is preferably one configured to vent gas from the through-bore 30, for example via a porous layer as described above.

Alternatively, a method of convection-enhanced delivery of an infusate to brain parenchyma may comprise implanting a guide tube 4 into the brain, wherein inserting the guide tube 4 into the brain comprises inserting the guide tube 4 with a fluid transfer tube 6 within the through-bore 30, and delivering the infusate into the brain via the fluid transfer tube. This is particularly suited for a guide tube 4 such as shown in FIG. 5 which is rigid and does not require a pre-formed guide tube track to be formed before it is inserted. Preferably a positive pressure of the infusate is provided within the fluid transfer tube 6 during insertion of the guide tube 4. This helps to prevent coring of brain tissue by the fluid transfer tube 6, and also prevents entraining of air that could occur if the fluid transfer tube 6 was inserted while filled with gas.

Each one of the guide tube 4, delivery probe, track making probe, and the fluid transfer tube may include any of the features described herein with respect to the other aspects.

Details of the method may also be described by the following numbered clauses.
M1. A method for implantation of a guide tube for convection-enhanced delivery of an infusate to brain parenchyma, the method comprising: inserting the guide tube into the brain until a distal end of the guide tube reaches a planned location in the brain, wherein the guide tube comprises a through-bore for passage of a fluid transfer tube and a distal end comprising a sealing region; compressing brain tissue adjacent to said sealing region to form a reflux-inhibiting seal around the distal end of the guide tube; forming a reduced-pressure region in which the compression of the brain tissue is lower than the compression of brain tissue in both a proximally adjacent region and a distally adjacent region to the reduced-pressure region.
M2. The method of clause M1, further comprising, prior to inserting the guide tube into the brain, inserting a probe into the brain parenchyma to form a guide tube track extending to the planned location, wherein: the guide tube has a diameter at least that of the probe; and inserting the guide tube into the brain comprises inserting the guide tube into the guide tube track.
M3. A method of convection-enhanced delivery of an infusate to brain parenchyma comprising: implanting a guide tube in the brain using the method of clause M1 or M2; advancing a delivery probe through the through-bore along the axis of the guide tube to form a fluid transfer tube track extending from the distal end of the guide tube; passing a fluid transfer tube through the through-bore of the guide tube and into the brain along the fluid transfer tube track; and delivering the infusate into the brain via the fluid transfer tube.
M4. A method of convection-enhanced delivery of an infusate to brain parenchyma comprising: implanting a guide tube in the brain using the method of clause M1 or M2, wherein inserting the guide tube into the brain comprises inserting the guide tube with a fluid transfer tube within the through-bore, preferably wherein a positive pressure of the infusate is provided within the fluid transfer tube during insertion of the guide tube; and delivering the infusate into the brain via the fluid transfer tube.
M5. The method of clause M2 or any preceding clause dependent thereon, wherein the probe comprises: a rod having a distal end that is rounded or conical in shape; and a spike extending axially from the distal end of the rod, the spike having a narrower diameter than the rod and having an extreme distal end configured for dissecting brain tissue.
M6. The method of clause M3, wherein the inserting of the guide tube is carried out with the delivery probe within the through-bore, such that a distal end of the delivery probe extends to or just beyond the distal end of the guide tube.
M7. The method of clause M3 or any preceding clause dependent thereon, wherein the fluid transfer tube track has a narrower diameter than the guide tube track.
M8. The method of any of clauses M1-M7, further comprising cutting the guide tube to a desired insertion length with respect to a fixation point in the skull prior to insertion of the guide tube into the brain.

1-58. (canceled)

59. A guide tube for use with a fluid transfer tube for providing fluid access to the brain of a mammal, wherein the guide tube is configured for insertion into the brain, and the guide tube comprises:
a through-bore for passage of a fluid transfer tube; and
a distal end comprising a sealing region configured to compress surrounding brain tissue to form a reflux-inhibiting seal around the distal end of the guide tube, the sealing region comprising a reduced-pressure region in which the compression of the brain tissue is lower than the compression of brain tissue in both a proximally adjacent region and a distally adjacent region.

60. The guide tube of claim 59, wherein the sealing region comprises a plurality of reduced-pressure regions.

61. The guide tube of claim 60, wherein at least one of:
a) the plurality of reduced-pressure regions are equally-spaced along a length of the guide tube;
b) a spacing between the reduced-pressure regions is at most 2 mm, preferably at most 1.5 mm, more preferably at most 1 mm;
c) the reduced-pressure regions are provided over at least 5% of a length of the guide tube; and
d) the reduced-pressure regions are provided over at least 2 mm of a length of the guide tube.

62. The guide tube of claim 59, wherein at least one of:
a) at least one of the reduced-pressure regions is at most 20 mm away from an extreme distal end of the guide tube; and
b) at least one of the reduced-pressure regions is at most 25% of a length of the guide tube away from an extreme distal end of the guide tube
c) a material and/or thickness of the guide tube is such that the guide tube can be cut by hand using a knife; and
d) the guide tube is flexible.

63. The guide tube of claim 59, wherein the reduced-pressure region is provided by a groove in an outer surface of the guide tube or between protrusions from an outer surface of the guide tube.

64. The guide tube of claim 63, wherein at least one of:
a) the groove has a depth of at most 0.5 mm, or the protrusions have a height of at most 0.5 mm;
b) the groove has a depth of at least 0.1 mm, or the protrusions have a height of at least 0.1 mm.

65. The guide tube of claim 63, wherein the groove or protrusions extend around the entire circumference of the guide tube.

66. The guide tube of claim 59, wherein at least one of:
a) an outer surface of the guide tube is configured to make contact with brain tissue following insertion of the guide tube into the brain;
b) an outer surface of the guide tube comprises a lubricious coating, for example of Parylene or PTFE;
c) an extreme distal end of the guide tube is rounded, bullet-shaped, or conical;
d) the distal end of the guide tube has a diameter of at most 2.5 mm, preferably 1.8 mm, more preferably at most 1.6 mm, most preferably at most 1.3 mm; and
e) the through-bore of the guide tube has a diameter of between 0.2 mm and 1 mm, preferably between 0.4 mm and 0.7 mm, more preferably between 0.5 mm and 0.6 mm.

67. The guide tube of claim 59, wherein the guide tube comprises a rigid material, for example ceramic, metal, or rigid plastic.

68. The guide tube of claim 67, wherein the guide tube has a length of between 50 mm and 300 mm, preferably between 100 mm and 250 mm.

69. The guide tube of claim 67, wherein the guide tube comprises a proximal end configured to be guided and/or held by a stereoguide or robot guide during insertion of the guide tube into the brain and/or during delivery of an infusate into the brain using the guide tube.

70. The guide tube of claim 69, wherein a proximal end of the guide tube has a larger diameter than the distal end of the guide tube, optionally wherein the proximal end of the guide tube has a diameter of at most 7 mm, preferably at most 6 mm, more preferably at most 5 mm.

71. The guide tube of claim 59, wherein the guide tube comprises a porous layer to allow passage of air, the porous layer comprising a hydrophobic material, optionally a superhydrophobic material.

72. The guide tube of claim 71, wherein at least one of:
a) the porous layer comprises at least one of sintered polytetrafluoroethylene, sintered polyurethane, expanded polytetrafluoroethylene, silicone foam, polyurethane foam, shape memory polymer, microporous hollow extruded-polymer fibres, or electrospun polymer;
b) the porous layer comprises microporous hollow extruded-polymer fibres or electrospun polymer, and the microporous hollow extruded-polymer fibres and/or the electrospun polymer comprises at least one of polytetrafluoroethylene, polyvinylidene difluoride, polyurethane, polypropylene, and copolymers thereof; and
c) the porous layer is an outermost layer of the guide tube.

73. The guide tube of claim 71, wherein the porous layer comprises a non-homogeneous material having varying stiffness and/or a plurality of materials having different stiffnesses, optionally wherein the stiffness of the porous layer is greater at or near the through-bore.

74. The guide tube of claim 73, wherein the porous layer comprises at least an inner layer and an outer layer, wherein the inner layer has greater stiffness than the outer layer and the inner layer is configured to allow passage of air through the inner layer, for example by comprising a plurality of holes through the inner layer or by comprising a material porous to air.

75. The guide tube of claim 74, wherein the inner layer is an innermost layer of the guide tube and provides the surface of the through-bore.

76. The guide tube of claim 74, wherein at least one of:
a) the inner layer comprises at least one of: polyether ether ketone; nylon; polyurethane; polyester; a fluoropolymer such as polytetrafluoroethylene; a polymeric perfluoroether such as perfluoroalkoxy alkane, polyvinylidene difluoride, or fluorinated ethylene propylene; liquid crystal polymers; and mixtures or copolymers thereof; and
b) the inner layer is manufactured by a process comprising at least one of: sintering, extrusion with particulate leaching, micro-perforating a tube by drilling or by laser; weaving, braiding or electrospinning polymer fibres about a cylindrical former to form a tube of porous polymer material; and 3D printing a polymer in a porous form.

77. The guide tube of claim 71, wherein the guide tube further comprises a fluid-impermeable covering layer provided over the porous layer, optionally wherein the covering layer is lubricious, further optionally wherein the covering layer comprises a heat shrink tube.

78. The guide tube of claim 77, wherein the reduced pressure regions are provided by the covering layer, optionally wherein the covering layer comprises a heat shrink tube with a series of, optionally annular, thickenings or grooves along its axis applied over the porous layer.

79. The guide tube of claim 59, wherein the guide tube is flexible and at least a proximal portion of the guide tube is resiliently deformable along an axial direction.

80. The guide tube of claim 79, wherein:
the guide tube comprises at least an inner layer and an outer layer;
the inner layer has greater stiffness than the outer layer; and
the outer layer is resiliently deformable; and wherein either:
a) the inner layer does not extend into the proximal portion of the guide tube; or
b) in the proximal portion of the guide tube, the inner layer is configured to provide a spring.

81. The guide tube of claim 59, wherein a proximal end of the guide tube comprises an enlargement configured for securing in a burr hole in a skull.

82. The guide tube of claim 81, wherein the enlargement comprises an increased-diameter portion configured to engage with a guide hub for securing to the skull.

83. The guide tube of claim 82, wherein at least one of:
a) engagement of the increased-diameter portion of the guide tube with the guide hub forms a fluid seal between the guide tube and the guide hub;
b) the guide tube comprises a resiliently deformable outer layer, and engagement of the increased-diameter portion of the guide tube with the guide hub comprises compression of the outer layer;
c) the through-bore of the guide tube within the increased-diameter portion increases in diameter towards the proximal end of the increased-diameter portion.

84. The guide tube of claim 82, wherein the increased-diameter portion comprises a non-compliant core located inward of the outer layer, optionally wherein the increased diameter portion is an over-mould onto the stiffer inner layer.

85. The guide tube of claim 84, wherein the non-compliant core is configured to allow the passage of gas through the non-compliant core, optionally wherein the non-compliant core comprises an air porous material such as sintered polyurethane or PEEK and/or the non-compliant core comprises air venting channels.

86. The guide tube of claim 84, wherein:
the guide tube comprises a porous layer to allow passage of air, the porous layer comprising a hydrophobic material;
the porous layer comprises at least an inner layer and an outer layer, wherein the inner layer has greater stiffness than the outer layer and the inner layer is configured to allow passage of air through the inner layer; and at least one of:
a) the non-compliant core is located inward of the outer layer; and
b) the non-compliant core is located outward of the inner layer or the non-compliant core is formed integrally with the inner layer, optionally wherein the inner layer does not extend to an extreme proximal end of the increased-diameter portion, further optionally wherein the inner layer extends through at most 80%, optionally at most 60% of the increased-diameter portion.

87. A neurosurgical apparatus comprising:
the guide tube of claim 82; and
a guide hub for securing to the skull of a patient before insertion of the guide tube into the brain, the guide hub having a passage for the guide tube therethrough.

88. A neurosurgical apparatus comprising:
the guide tube of claim 59; and at least one of:
a) a fluid transfer tube, preferably a cannula, configured for insertion into the through-bore of the guide tube;
b) a probe configured for insertion into brain tissue, the probe comprising: a rod having a distal end that is rounded or conical in shape; and a spike extending axially from the distal end of the rod, the spike having a narrower diameter than the rod and having an extreme distal end configured for dissecting brain tissue; and
c) a delivery probe configured for insertion into the through-bore of the guide tube, wherein a distal end of the delivery probe is configured for dissecting brain tissue.

89. A method for implantation of a guide tube for convection-enhanced delivery of an infusate to brain parenchyma, the method comprising:
inserting the guide tube into the brain until a distal end of the guide tube reaches a planned location in the brain, wherein the guide tube comprises a through-bore for passage of a fluid transfer tube and a distal end comprising a sealing region;
compressing brain tissue adjacent to said sealing region to form a reflux-inhibiting seal around the distal end of the guide tube; and
forming a reduced-pressure region in which the compression of the brain tissue is lower than the compression of brain tissue in both a proximally adjacent region and a distally adjacent region to the reduced-pressure region.

90. The method of claim 89, further comprising at least one of:
a) prior to inserting the guide tube into the brain, inserting a probe into the brain parenchyma to form a guide tube track extending to the planned location, wherein: the guide tube has a diameter at least that of the probe; and inserting the guide tube into the brain comprises inserting the guide tube into the guide tube track; and
b) cutting the guide tube to a desired insertion length with respect to a fixation point in the skull prior to insertion of the guide tube into the brain.

91. A method of convection-enhanced delivery of an infusate to brain parenchyma comprising:
implanting a guide tube in the brain using the method of claim 89;
advancing a delivery probe through the through-bore along the axis of the guide tube to form a fluid transfer tube track extending from the distal end of the guide tube;
passing a fluid transfer tube through the through-bore of the guide tube and into the brain along the fluid transfer tube track; and
delivering the infusate into the brain via the fluid transfer tube.

92. A method of convection-enhanced delivery of an infusate to brain parenchyma comprising:
implanting a guide tube in the brain using the method of claim 89, wherein inserting the guide tube into the brain comprises inserting the guide tube with a fluid transfer tube within the through-bore, preferably wherein a positive pressure of the infusate is provided within the fluid transfer tube during insertion of the guide tube; and
delivering the infusate into the brain via the fluid transfer tube.

93. The method of claim 90, wherein the probe comprises:
a rod having a distal end that is rounded or conical in shape; and
a spike extending axially from the distal end of the rod, the spike having a narrower diameter than the rod and having an extreme distal end configured for dissecting brain tissue.

94. The method of claim 91, wherein at least one of:
a) the inserting of the guide tube is carried out with the delivery probe within the through-bore, such that a distal end of the delivery probe extends to or just beyond the distal end of the guide tube; and
b) the fluid transfer tube track has a narrower diameter than the guide tube track.
